(19) United States
(12) Patent Application Publication
(10) Pub. No.: US 2020/0222391 A1
(43) Pub. Date: Jul. 16, 2020

(54) SOLID PREPARATION OF CARIPRAZINE FOR ORAL ADMINISTRATION

(71) Applicant: Richter Gedeon Nyrt., Budapest (HU)

(72) Inventors: Melinda Konta, Budapest (HU); Edit Suba, Marosvasarhely (RO); Tunde Beata Daroczi, Gyuro (HU); Zoltan Magos, Budapest (HU); Ramona Rajszkine Labos, Budapest (HU)

(21) Appl. No.: 16/622,020

(22) PCT Filed: Jun. 12, 2018
(86) PCT No.: PCT/IB2018/054227

§ 371 Date: Dec. 12, 2019

(30) Foreign Application Priority Data
Jun. 13, 2017 (HU) P1700253

(51) Int. Cl. A61K 31/495; A61K 9/00; A61K 47/38; A61K 9/48

(52) U.S. Cl. CPC A61K 31/495; A61K 9/4808; A61K 47/38; A61K 9/0053

(57) ABSTRACT

The invention relates oral pharmaceutical compositions for the modified release delivery of cariprazine (trans-N-{4[2-[4-(2,3-dichlorophenyl)-piperazin-1-yl]-ethyl]-cyclohexyl}-N′,N′-dimethylurea) or pharmaceutically acceptable salts thereof for less than daily dosing. The invention also relates to the use of said compositions in the treatment and/or prevention of pathological conditions which require the modulation of dopamine receptors. The invention also relates to the process for the preparation of said modified release pharmaceutical compositions.

### CROSS-REFERENCE TO RELATED APPLICATIONS

This application is a National Stage application under 35 U.S.C. § 371 of International Application No. PCT/IB2018/054227, having an International Filing Date of Jun. 12, 2018, which claims benefit of priority from HU Application No. P1700253, filed on Jun. 13, 2017. The disclosures of the prior applications are considered part of (and are incorporated by reference in) the disclosure of this application.

### FIELD OF THE INVENTION

The present invention provides oral pharmaceutical compositions and methods for the modified release delivery of cariprazine (trans-N-{4-[2-[4-(2,3-dichlorophenyl)-piperazin-1-yl]-ethyl]-cyclohexyl}-N′,N′-dimethylurea) or pharmaceutically acceptable salts thereof for less than daily dosing.

### BACKGROUND OF THE INVENTION

Cariprazine is a dopamine D3-preferring D3/D2 receptor partial agonist. Document WO 2005/012266 A1 discloses cariprazine and its pharmaceutically acceptable salts. This document also discloses pharmaceutical compositions containing hydrochloride or other pharmaceutically acceptable salts of cariprazine and their use for therapy and/or prevention of pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, autism.

Cariprazine produces two clinically relevant metabolites: desmethyl-cariprazine (DCAR) and didesmethyl-cariprazine (DDCAR). As the in vitro receptor profile and potency of the metabolites are similar to those of the parent compound, and the plasma protein binding as well as the brain penetration of cariprazine, DCAR and DDCAR are also similar, the plasma exposures of the moieties directly reflect their contribution to the in vivo pharmacological effects of the medicinal product. All these compounds should be considered together as the active pharmaceutical ingredient of the medicinal product.

Presently, only immediate release (IR) preparations of cariprazine hydrochloride are available for use as a medicament. WO 2010/009309 A1 discloses stable and bioavailable immediate release pharmaceutical compositions of the drug. According to WO 2009/104739 A1 a solid preparation for oral administration of cariprazine hydrochloride has been developed as a new immediate release tablet dosage form. Further immediate release dosage forms of cariprazine hydrochloride, in particular granules, fine granules or powders having superior properties are described in EP 16165247 A1.

The presently available use of the solid dosage forms of cariprazine and its pharmaceutically acceptable salts is restricted to daily administration due to their immediate release character. The longer the patients have to administer their medicaments the higher is the need for a less frequent dosage regimen, since the efficient long term therapy is closely related to the patients' compliance, particularly for patients treated with different central nervous system (CNS) diseases, including schizophrenia.

Several studies directly relate nonadherence with higher rates of relapse, increased number of re-hospitalizations, increased dependence on families and on the healthcare system, and worsening of long-term prognosis and functionality.

According to the prior art, there are several mechanisms in general controlling drug release including dissolution, partitioning, diffusion, osmosis, swelling, erosion, and targeting. [J. Siepmann et al. (eds.), Fundamentals and Applications of Controlled Release Drug Delivery, Advances in Delivery Science and Technology, DOI 10.1007/978-1-4614-0881-9_2, # Controlled Release Society 2012]. The mode of controlled drug delivery depends on the particular application and some of them could be combined and take part together or at different stages of the final controlling mechanism.

The prior art discloses several different mechanisms to decrease the dosing frequency of antipsychotic drugs, such as modified release oral formulations and long-acting injectable compositions.

WO 2008/038003 A1 discloses controlled release oral pharmaceutical compositions comprising aripiprazole. The compositions may be formulated as a diffusion-controlled formulation, a dissolution-controlled formulation, an easily administrable formulation, an enteric-coated formulation, an osmotic pump technology formulation, a tamper-resistant formulation, an erosion-controlled formulation, an ion exchange resin or a combination of the foregoing.

U.S. Pat. No. 5,910,319 B1 patent discloses enteric formulations of fluoxetine, in the form of enteric pellets, which comprise a core consisting of fluoxetine and one or more pharmaceutically acceptable excipients; an optional separating layer comprising a non-reducing sugar and one or more pharmaceutically acceptable excipients; an enteric layer comprising hydroxypropylmethylcellulose acetate succinate and one or more pharmaceutically acceptable excipients; and an optional finishing layer.

The development of long-acting injectable (LAI) antipsychotics is a pharmacological strategy for treating patients with schizophrenia who relapse due to nonadherence to antipsychotic medication, as the LAI antipsychotics are administered by injection at two to four week intervals, differently from the daily administration of oral antipsychotics. These antipsychotic drugs are marketed in long-lasting forms: aripiprazole (Abilify Maintena); aripiprazole lauroxil (Aristada), fluphenazine (Prolixin); haloperidol (Haldol); olanzapine pamoate (Zyprexa Relprevv); paliperidone (Invega Sustenna, Invega Trinza) and risperidone (Risperdal Consta).

In addition to the known advantages of long-acting injectable antipsychotics, there are a number of disadvantages that need to be considered regarding clinical practice. Some of these are more relevant, such as perception of stigma, pain at the injection site, and leakage into the subcutaneous tissue and/or the skin causing irritation and lesions (especially for oily long-acting injectables), and higher manufacturing costs.

Therefore, there is a need to develop orally administrable non-immediate release (modified release) pharmaceutical formulations of cariprazine and its pharmaceutically acceptable salts, which are capable of reducing the dosing frequency, have the bioavailability values that enable this composition for a less frequent than daily administration, which is an efficient, cost-effective and convenient tool for a lifelong therapy and/or prevention of pathological conditions listed above.

The development of new compositions that allow greater quantities of cariprazine to be administered in one dose without significantly increasing the adverse effects compared to the current once daily conventional immediate release (IR) dosage regimen is required. Reduction in dosing frequency offers significant pharmacoeconomic advantages over the present dosage regimen by reducing the indirect human cost of the treatment (e.g. by reducing medical practitioners' time required for supervised drug administration).

In a further aspect, it is required to develop modified release pharmaceutical formulations which meet the regulation requirements of “dose dumping”, to improve the patient compliance and to reduce the side effects through more consistent plasma levels, leading to more effective therapies. “Dose dumping” refers to the rapid release of the entire dose or a significant fraction thereof in a short period of time.

Dose dumping resulting from consumption of alcoholic beverages in timely connection with the administration of a medication is referred to as “alcohol-induced dose dumping”. Specific patient populations, such as people with mental disorder characterized by abnormal social behaviour have the tendency to turn to alcohol as a way to cope with their conditions. People with schizophrenia often have additional mental health problems, such as anxiety disorders, major depressive illness or substance use disorders. As drug release is modified, dose dumping may occur if the release control is compromised through dissolution of the controlling agent in hydro-alcoholic liquids. [Regulatory Considerations for Alcohol-Induced Dose Dumping of Oral Modified-Release Formulations, Pharmaceutical Technology, Volume 38, Issue 10, pp 40-46]

Therefore, the modified release composition has to provide a safe use for patients who are consuming hydro-alcoholic liquids during the treatment period.

Additionally, there is also a need to provide simple methods of preparation that can be scaled to industrial level and the manufacture has to be economically feasible for a long term.

Particularly, a modified release product is capable to maintain the efficient dose at a precise controlled rate which is in mass balance with the rate of drug elimination corresponding to the required therapeutic concentration of drug in the plasma, without any adverse effect; and it is also capable to achieve a therapeutic concentration of cariprazine promptly in the body and then to maintain that concentration for a given period of time.

Our aim was to achieve satisfactory tolerability and convenient dosing in the long term therapy in a cost-effective manner. It is well known, that a modified release composition as an oral depot formulation ensures a less frequent dosage regimen and it is suitable to provide a favourable pharmacokinetic profile. In order to obtain it, the pharmacokinetic properties of the drug have to be studied comprehensively.

Pharmacokinetics describe how the body affects a drug after administration through the mechanisms of absorption and distribution, as well as the metabolic changes of the substance in the body, and the effects and routes of excretion of the metabolites of the drug. Pharmacokinetic properties of chemicals are affected by the route of administration and the dose of administered drug. These may affect the absorption rate. [In Mosby's Dictionary of Medicine, Nursing & Health Professions. Philadelphia, Pa.: Elsevier Health Sciences. Retrieved Dec. 11, 2008, from http://www.credoreference.com/entry/6686418; Jump up ̂ Kathleen Knights; Bronwen Bryant (2002). Pharmacology for Health Professionals. Amsterdam: Elsevier. ISBN 0-7295-3664-5].

In order to develop a modified release oral pharmaceutical composition it is necessary to consider the physiology of the gastrointestinal tract, the physicochemical properties of the active substance, the design of the dosage form, the drug release mechanism and the biological properties of the drug.

For a drug to be absorbed it needs to be in solution at first, and secondly, it has to pass across the membrane; which is the gastrointestinal epithelium in case of orally administered drugs.

Dissolution rate of the drug or other ingredients in the gastrointestinal fluids must be taken into account during the development. It is known that the environment within the lumen of the gastrointestinal tract has a major effect on the rate and extent of drug dissolution and absorption.

The residence time of a modified release delivery system in the gastrointestinal tract is a key factor aiming a desired bioavailability, and it is influenced by both stomach emptying time and intestinal transit time.

Dissolution is the transfer of molecules or ions from solid state into solution. The extent to which the dissolution proceeds under a given set of experimental conditions is referred to as the solubility of the solute in the solvent. Thus, the solubility of a substance is the amount of it that passes into solution when equilibrium is established between the solution and excess (undissolved) substance. [Pharmaceutics, The science of dosage form design (2002); Chapter 1/p16]. The absorption is the movement of a drug into the bloodstream.

The transit time, among several features of human gastrointestinal tract, might be very variable. Therefore, it is necessary to select suitable excipients in order to provide the desired drug release and absorption.

A number of physiological factors, such as gastrointestinal pH, enzyme activities, gastric and intestinal transit rates, food or any kind of gastrointestinal disease which often influence drug bioavailability from conventional oral dosage forms may also interfere with the dissolution and absorption of drugs from the oral modified release forms. Furthermore, the rate of the transit of modified release oral products along the gastrointestinal tract limits the maximum period for which a therapeutic response can be maintained following administration of a single dose to approximately 12 hours. Moreover, the length of time that absorbed drug continues to exert its therapeutic activity should be taken into account. [Pharmaceutics, The science of dosage form design (2002); Chapter 20/p294]

Additionally, the solubility profile of the active compound through the gastrointestinal tract has to be considered as well. Particularly, the pH of fluids varies considerably along the length of the gastrointestinal tract.

There is a natural pH gradient from the acidity of the stomach through the weakly acidic duodenum to the virtually neutral environment of the small intestine where the pH is in the range of 5-8. Gastric fluid is highly acidic; it is specified within the range 1-3.5 in healthy people in the fasted state and following the ingestion of a meal the gastric juice is buffered to less acidic pH. Typical gastric pH values following a meal are in the range of 3-7. Intestinal pH values are higher than gastric pH values owing to the neutralization of the gastric acid with bicarbonate ions secreted by the pancreas into the small intestine. There is a gradual rise in pH along the length of the small intestine from the duodenum to ileum [Pharmaceutics, The science of dosage form design (2002); Chapter 16/p224-p 225].

All drugs exhibit at least limited aqueous solubility for therapeutic efficiency. Thus, relatively insoluble compounds can exhibit erratic or incomplete absorption, and it might be appropriate to use more soluble salts or other chemical derivatives. Solubility, and especially degree of saturation in the vehicle, can also be important in the absorption of drugs already in solution in liquid dosage forms, as precipitation in the gastrointestinal tract can occur and bioavailability can be modified.

The solubility of acidic or basic compounds are pH-dependent and can be altered by forming salt forms with different salts exhibiting different equilibrium solubility. However, the solubility of a salt of strong acid is less affected by changes in pH than the solubility of a salt of week acid. In the latter case, when pH is lower the salt hydrolyses to an extent dependent on pH and pKa, resulting in decreased solubility. Reduced solubility can also occur for slightly soluble salts of drugs through the common ion effect. If one of the ions involved is added as a different, more water-soluble salts, the solubility product can be exceeded and a portion of the drug precipitates [Pharmaceutics, The science of dosage form design; (2002) Chapter 1/p7].

If the pH of a solution of either a weakly acidic drug or salt of such a drug is reduced then the proportion of unionized acid molecules in the solution increases. Precipitation may therefore occur, because the solubility of the unionized species is less than that of the ionized form. Conversely, in the case of solutions of weakly basic drugs or their salts precipitation is favoured by an increase in pH. This relationship between pH and the solubility of ionized solutes is extremely important with respect to the ionization of weakly acidic and basic drugs as they pass through the gastrointestinal tract and experience pH changes between about 1 and 8. This will affect the degree of ionization of the drug molecules, which in turn influences their solubility and their ability to be absorb. [Pharmaceutics, The science of dosage form design (2002); Chapter 1/p27].

Salts are formed when a compound that is ionized in solution forms a strong ionic interaction with an oppositely charged counterion, leading to crystallization of the salt form. All acidic and basic compounds can participate in salt formation.

Salt formation offers many advantages to the pharmaceutical products as it can improve the solubility, dissolution rate, permeability, and efficacy of the drug. The primary purpose of forming a salt is to increase the amount of drug in solution. Salt forms of drugs have significant effects on physicochemical properties of the drug influencing its quality, safety, and performance.

Importantly, different salt forms rarely change drug's pharmacological properties. The total concentration rises with a decrease in pH for the weak base, whereas it rises with an increase in pH for the weak acid.

Cariprazine salts are very well soluble in acidic environment. However, drugs, which are soluble in acidic environment can be practically insoluble in neutral or basic environment. This is in line with the fact that cariprazine hydrochloride has a solubility of 3.258 mg/ml at pH 1 and a solubility of 0.001 mg/ml at pH 7. According to the solubility study, cariprazine hydrochloride shows the best solubility around the pH value of 3. The values measured at 37° C. demonstrate pH-dependent solubility character of cariprazine hydrochloride.

TABLE 1
pH-solubility of cariprazine hydrochloride at 37° C.

| | Solubility | | Solubility |
|---|---|---|---|
| pH | [mg/ml] | pH | [mg/ml] |
| 1 | 3.2579 | 5 | 0.3510 |
| 2 | 8.9336 | 5.5 | 0.1488 |
| 3 | 11.0321 | 6 | 0.0188 |
| 4 | 3.2303 | 7 | 0.0013 |

The dissolution profile of the immediate release compositions (described in Example 4) correspond with the solubility of cariprazine hydrochloride, since over pH 5.5 the dissolution of the drug decreases significantly. Moreover, the presence of surfactants in the biorelevant is dissolution media—which simulates gut fluids before (Fasted-State Simulated Intestinal Fluid (FaSSIF)) and after (Fed-State Simulated Intestinal Fluid (FeSSIF)) eating food—do not raise the dissolution of cariprazine at higher pH values (see tables 2 and 3).

TABLE 2
Dissolution profile of the cariprazine 2.5 mg IR capsules

| | |
|---|---|
| cariprazine 2.5 mg IR | |
| capsules | |
| (Batch No. 0A05) | Dissolution rate (%) |

| Time (min) | 5 | 10 | 15 | 30 |
|---|---|---|---|---|
| 0.001n HCl | 52 | 98 | 101 | 102 |
| pH = 4.5 buffer | 47 | 93 | 96 | 97 |
| pH = 5.0 buffer | 43 | 92 | 96 | 97 |
| pH = 5.5 buffer | 38 | 82 | 86 | 88 |
| pH = 6.0 buffer | 27 | 71 | 79 | 87 |
| pH = 6.8 buffer | 11 | 28 | 35 | 47 |
| pH = 6.8 buffer FeSSIF | 56 | 82 | 84 | 87 |
| pH = 6.8 buffer FaSSIF | 12 | 48 | 56 | 64 |

TABLE 3
Dissolution profile of the cariprazine 25 mg IR capsules

| | |
|---|---|
| cariprazine 25 mg IR | |
| capsules | Dissolution rate (%) |

| (Batch No. 0A04) | 5 | 10 | 15 | 30 |
|---|---|---|---|---|
| 0.001n HCl | 51 | 92 | 94 | 94 |
| pH = 4.5 buffer | 57 | 88 | 90 | 92 |
| pH = 5.0 buffer | 53 | 86 | 88 | 89 |
| pH = 5.5 buffer | 42 | 80 | 83 | 87 |
| pH = 6.0 buffer | 30 | 60 | 65 | 67 |
| pH = 6.8 buffer | 3 | 7 | 8 | 12 |
| pH = 6.8 buffer FeSSIF* | 35 | 61 | 62 | 67 |
| pH = 6.8 buffer FaSSIF* | 16 | 27 | 31 | 34 |

Accordingly, it is not evidently feasible to produce compositions which enable appropriate control on the drug's release through the whole gastrointestinal tract.

The solubility is the factor that mainly determines the bioavailability of cariprazine, as it exhibits high permeability according to Caco-2 studies. In the Caco-2 model of drug absorption, the permeability coefficients of cariprazine in inward and outward directions were calculated to be 26.4.10-6 cm/sec and 51.2.10-6 cm/sec, respectively (permeability directional ratio (PDR): 1.9) (Artursson P & Karlsson J (1991). “Correlation between oral drug absorption in humans and apparent drug permeability coefficients in human intestinal epithelial (Caco-2) cells”. Biochem Biophys Res Comm 175 (3): 880-5 and internal data).

Consequently, it is evident that the release characteristics can be modified by the composition and it highly depends on the solubility profile of the active substance.

Therefore, the drug release of cariprazine hydrochloride from conventional systems containing only pH-independent swelling polymers is expected to be much faster in the stomach compared to the slower or even incomplete drug release in the small intestine and the colon.

In order to find a suitable delivery system for cariprazine and its pharmaceutically acceptable salts a number of formulations have been prepared and evaluated.

Consequently, the objective of the present invention is to provide oral pharmaceutical compositions comprising cariprazine salts with at least one release-modifying agent suitable for decreasing the Cₘₐₓ and keeping the AUC values within the range of the effective and tolerable therapeutic daily doses aiming at an elongated effect in the desired administration frequency independently of the location of the drug release in the gastrointestinal tract.

### BRIEF DESCRIPTION OF THE DRAWINGS

An exemplary embodiment of the present invention is illustrated by way of example in the accompanying drawings in which like reference numbers indicate the same or similar elements and in which:

FIG. 1 illustrates mean cariprazine plasma concentrations (μg/mL) following single oral administration of the IR, PR A and PR B compositions according to Example 13.

FIG. 2 illustrates the steady-state simulations of cariprazine following oral administration of 6 mg of PR B every 4 days according to Example 14.

FIG. 3 illustrates the steady-state simulations of cariprazine following oral administration of 10.5 mg of PR B every 7 days according to Example 14.

FIG. 4 illustrates the steady-state simulations of cariprazine following oral administration of 12 mg of PR B every 4 days according to Example 14.

FIG. 5 illustrates the steady-state simulations of cariprazine following oral administration of 18 mg of PR B every 4 days according to Example 14.

FIG. 6 illustrates the steady-state simulations of cariprazine following oral administration of 21 mg of PR B every 14 days according to Example 14.

FIG. 7 illustrates the steady-state simulations of cariprazine following oral administration of 24 mg of PR B every 4 days according to Example 14.

FIG. 8 illustrates the steady-state simulations of cariprazine following oral administration of 42 mg of PR B every 7 days according to Example 14.

### SUMMARY OF THE INVENTION

Cariprazine salts are very well soluble in acidic environment, and the prior art teaches, that micro-environmental pH modulation or solubility enhancement is essential to achieve the complete dissolution of active ingredients characterized by pH-dependent solubility from modified release pharmaceutical compositions. However, during the development it was surprisingly found, that these complicated methods are completely unnecessary, and simple matrix tablet formulations provide favourable pharmacokinetic profile, as they are able to decrease the Cₘₐₓ and keep the AUC values within the range of the effective and tolerable therapeutic daily doses.

The invention relates to orally deliverable solid pharmaceutical compositions for the modified release of cariprazine or pharmaceutically acceptable salts thereof, wherein the composition comprises a therapeutically effective amount of cariprazine or a pharmaceutically acceptable salt thereof and at least one release-modifying agent.

The present invention also relates to the pharmaceutical compositions as defined above for use in the treatment and/or prevention of pathological conditions which require the modulation of dopamine receptors, wherein the treatment and/or prevention comprises the administration of the pharmaceutical compositions less frequent than once daily.

The present invention also relates to the use of the pharmaceutical composition as defined above in the manufacture of a medicament for the treatment and/or prevention of pathological conditions which require the modulation of dopamine receptors, wherein the treatment and/or prevention comprises administration of the pharmaceutical compositions less frequent than once daily.

The present invention also relates to the process for the preparation of modified release pharmaceutical compositions as defined above in different dosage forms, wherein the compositions are obtained by conventional methods known in the art, including direct compression of the ingredients into tablets, and optionally coating them; fluid granulation and thereafter compression; and extrusion and spheronization of the ingredients and thereafter filling the obtained spheres into capsules.

The present invention also relates to the method of treating a patient suffering from pathological conditions which require the modulation of dopamine receptors, wherein the method comprises the administration of the pharmaceutical compositions as defined above.

### DETAILED DESCRIPTION OF THE INVENTION

The present invention provides orally deliverable solid pharmaceutical compositions for the modified release of cariprazine and its pharmaceutically acceptable salts for the treatment and/or prevention of pathological conditions which require the modulation of dopamine receptors, which comprises a therapeutically effective amount of the active ingredient and at least one release-modifying agent.

Particularly, the invention relates to orally deliverable solid pharmaceutical compositions for the modified release of cariprazine or pharmaceutically acceptable salts thereof, wherein the composition comprises a therapeutically effective amount of cariprazine or a pharmaceutically acceptable salt thereof and at least one release-modifying agent suitable for decreasing the Cₘₐₓ and keeping the AUC values within the range of the effective and tolerable therapeutic daily doses aiming at an elongated effect in the desired administration frequency independently of the location of the drug release in the gastrointestinal tract.

In a preferred embodiment, the invention provides a solid pharmaceutical composition comprising from about 1.5 mg to about 84 mg, including about 1.5 mg, about 3 mg, about 4.5 mg, about 6 mg, about 9 mg, about 10.5 mg, about 12 mg, about 15 mg, about 18 mg, about 21 mg, about 24 mg, about 27 mg, about 30 mg, about 31.5 mg, about 42 mg, about 60 mg, about 63, or about 84 mg cariprazine in the form of a pharmaceutically acceptable salt.

In a more preferred embodiment, the invention provides a solid pharmaceutical composition comprising from about 1.5 mg to about 31.5 mg, including about 1.5 mg, about 3 mg, about 4.5 mg, about 6 mg, about 9 mg, about 10.5 mg, about 12 mg, about 15 mg, about 18 mg, about 21 mg, about 24 mg, about 27 mg, about 30 mg, or about 31.5 mg cariprazine in the form of a pharmaceutically acceptable salt.

In a particularly preferred embodiment, the invention provides a solid pharmaceutical composition comprising from about 1.5 mg to about 24 mg, including about 1.5 mg, about 3 mg, about 4.5 mg, about 6 mg, about 9 mg, about 10.5 mg, about 12 mg, about 15 mg, about 18 mg, about 21 mg or about 24 mg, cariprazine in the form of a pharmaceutically acceptable salt.

In the most preferred embodiment, the invention provides a solid pharmaceutical composition comprising from about 1.5 mg to about 12 mg, including about 1.5 mg, about 3 mg, about 4.5 mg, about 6 mg, about 9 mg, about 10.5 mg, or about 12 mg cariprazine in the form of a pharmaceutically acceptable salt.

In a further preferred embodiment of the present invention the solid pharmaceutical composition contains more than 1.5 mg cariprazine in the form of a pharmaceutically acceptable salt.

In a further preferred embodiment of the present invention the solid pharmaceutical composition contains at most 84 mg cariprazine in the form of a pharmaceutically acceptable salt.

In a further preferred embodiment, the present invention provides a solid pharmaceutical composition comprising from about 6 mg to about 30 mg cariprazine in the form a pharmaceutically acceptable salt.

In a preferred embodiment, the present invention provides a solid pharmaceutical composition comprising from about 6 mg to about 24 mg cariprazine in the form of a pharmaceutically acceptable salt.

In a preferred embodiment, the present invention provides a solid pharmaceutical composition comprising from about 1.5 mg to about 84 mg cariprazine in the form of hydrochloride salt.

In a more preferred embodiment, the present invention provides a solid pharmaceutical composition comprising from about 6 mg to about 30 mg cariprazine in the form of hydrochloride salt.

In the most preferred embodiment, the present invention provides a solid pharmaceutical composition comprising from about 6 mg to about 24 mg cariprazine in the form of hydrochloride salt.

In a preferred embodiment of the present invention the solid pharmaceutical composition comprises a pharmaceutically acceptable salt of cariprazine selected from the group comprising a salt of hydrochloric acid, sulphuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid.

In a more preferred embodiment of the present invention the solid pharmaceutical composition comprises a pharmaceutically acceptable salt of cariprazine selected from the group comprising a salt of hydrochloric acid, hydrobromic acid and methanesulfonic acid.

In the most preferred embodiment of the present invention the solid pharmaceutical composition comprises cariprazine hydrochloride.

In a preferred embodiment of the present invention the solid pharmaceutical composition comprises at least one release-modifying agent selected from the group comprising hydrophilic and hydrophobic polymers.

In a more preferred embodiment of the present invention the solid pharmaceutical composition comprises at least one hydrophilic polymer as a release-modifying agent.

In a more preferred embodiment of the present invention the solid pharmaceutical composition comprises at least one cellulose-based polymer as a release-modifying agent.

In a more preferred embodiment of the present invention the solid pharmaceutical composition comprises at least one cellulose-based polymer as a release-modifying agent such as hydroxyalkyl celluloses selected from the group comprising hydroxypropyl cellulose (HPC), hydroxyethyl cellulose (HEC), hydroxymethyl cellulose and hydroxypropyl methylcellulose (HPMC), carboxymethyl cellulose, sodium carboxymethyl cellulose, methylcellulose, and hydroxyethyl methylcellulose.

In the most preferred embodiment of the present invention the solid pharmaceutical composition comprises at least one cellulose-based polymer as a release-modifying agent such as hydroxyalkyl celluloses selected from the group comprising hydroxypropyl cellulose (HPC), hydroxyethyl cellulose (HEC), hydroxymethyl cellulose and hydroxypropyl methylcellulose (HPMC).

In a more preferred embodiment of the present invention the solid pharmaceutical composition comprises at least one hydrophobic polymer as a release-modifying agent.

In a preferred embodiment of the present invention the solid pharmaceutical composition comprises at least one release-modifying agent from about 15 to about 75% by weight.

In a more preferred embodiment of the present invention the solid pharmaceutical composition comprises at least one release-modifying agent from about 25 to about 65% by weight.

In a preferred embodiment of the present invention the solid pharmaceutical composition as defined above further comprises other excipients alone or in any combination, selected from the group of diluents, lubricants, effervescent components, binders, granulating aids, film formers, and glidants.

In a preferred embodiment of the present invention the solid pharmaceutical composition is designed for oral administration, including, but not limited to tablets, capsules, granules, powders, microspheres, pellets, and beads.

In a preferred embodiment, the invention relates to a pharmaceutical composition comprising cariprazine, which provides a dissolution profile, wherein about 25% to about 70% of the total amount of cariprazine is in solution at 4 hours, and about 45% to about 100% of the total amount of cariprazine is in solution at 8 hours, about 65% to about 100% of the total amount of cariprazine is in solution at 12 hours.

In a more preferred embodiment, the invention relates to a pharmaceutical composition comprising cariprazine, which provides a dissolution profile, wherein about 30% to about 65% of the total amount of cariprazine is in solution at 4 hours, and about 50% to about 95% of the total amount of cariprazine is in solution at 8 hours, and about 70% to about 100% of the total amount of cariprazine is in solution at 12 hours.

In the most preferred embodiment, the invention relates to a pharmaceutical composition comprising cariprazine, which provides a dissolution profile, wherein about 35% to about 60% of the total amount of cariprazine is in solution at 4 hours, and about 55% to about 90% of the total amount of cariprazine is in solution at 8 hours, and 75% to about 100% of the total amount of cariprazine is in solution at 12 hours.

In another preferred embodiment of the present invention the pharmaceutical composition as defined above exhibits a cariprazine AUC value following oral administration that is from about 60% to about 145% of that achieved using an immediate release (IR) dosage form of cariprazine when administered orally at an equivalent dose.

In a more preferred embodiment of the present invention the pharmaceutical composition as defined above has exhibits a cariprazine AUC value following oral administration that is from about 70% to about 135% of that achieved using an immediate release (IR) dosage form of cariprazine when administered orally at an equivalent dose.

In a more preferred embodiment of the present invention the pharmaceutical composition as defined above exhibits a cariprazine AUC value following oral administration that is from about 80% to about 125% of that achieved using an immediate release (IR) dosage form of cariprazine when administered orally at an equivalent dose.

In another more preferred embodiment of the present invention the pharmaceutical composition as defined above exhibits a cariprazine AUC value following oral administration that is from about 90% to about 115% of that achieved using an immediate release (IR) dosage form of equivalent dose is administered.

In the most preferred embodiment of the present invention the pharmaceutical composition defined above exhibits a cariprazine AUC value following oral administration that is from about 95% to about 105% of that achieved when an immediate release (IR) dosage form of cariprazine when administered orally at an equivalent dose.

In another preferred embodiment, the invention relates to a pharmaceutical composition comprising cariprazine, which provides a PK profile after oral administration in a human wherein Cₘₐₓ is from about 8% to about 40% of the Cₘₐₓ obtained by an IR formulation comprising the same amount of cariprazine as said modified release pharmaceutical composition; when said PK profile arises from a PK experiment performed in a human fasted overnight for at least eight hours prior to dosing; wherein said PK profile is based on plasma concentrations of the sum of cariprazine parent and des- and didesmethyl-cariprazine; and wherein said pharmaceutical composition comprises cariprazine in a therapeutically effective amount.

In a more preferred embodiment, the invention relates to a pharmaceutical composition comprising cariprazine, which provides a PK profile after oral administration in a human wherein Cₘₐₓ is from about 8% to about 35% of the Cₘₐₓ obtained by an IR formulation comprising the same amount of cariprazine as said modified release pharmaceutical composition; when said PK profile arises from a PK experiment performed in a human fasted overnight for at least eight hours prior to dosing;

wherein said PK profile is based on plasma concentrations of the total cariprazine; and wherein said pharmaceutical composition comprises cariprazine in a therapeutically effective amount.

In a more preferred embodiment, the invention relates to a pharmaceutical composition comprising cariprazine, which provides a PK profile after oral administration in a human wherein Cmax is from about 8% to about 30% of the Cₘₐₓ obtained by an IR formulation comprising the same amount of cariprazine as said modified release pharmaceutical composition; when said PK profile arises from a PK experiment performed in a human fasted overnight for at least eight hours prior to dosing; wherein said PK profile is based on plasma concentrations of the total cariprazine; and wherein said pharmaceutical composition comprises cariprazine in a therapeutically effective amount.

In a further more preferred embodiment, the invention relates to a pharmaceutical composition comprising cariprazine, which provides a PK profile after oral administration in a human wherein Cₘₐₓ is from about 8% to about 25% of the Cₘₐₓ obtained by an IR formulation comprising the same amount of cariprazine as said modified release pharmaceutical composition; when said PK profile arises from a PK experiment performed in a human fasted overnight for at least eight hours prior to dosing; wherein said PK profile is based on plasma concentrations of the total cariprazine; and wherein said pharmaceutical composition comprises cariprazine in a therapeutically effective amount.

In a further more preferred embodiment, the invention relates to a pharmaceutical composition comprising cariprazine, which provides a PK profile after oral administration in a human wherein Cₘₐₓ is from about 8% to about 20% of the Cₘₐₓ obtained by an IR formulation comprising the same amount of cariprazine as said modified release pharmaceutical composition; when said PK profile arises from a PK experiment performed in a human fasted overnight for at least eight hours prior to dosing; wherein said PK profile is based on plasma concentrations of total cariprazine; and wherein said pharmaceutical composition comprises cariprazine in a therapeutically effective amount.

In a most preferred embodiment, the invention relates to a pharmaceutical composition comprising cariprazine, which provides a PK profile after oral administration in a human wherein Cₘₐₓ is from about 8% to about 15% of the Cₘₐₓ obtained by an IR formulation comprising the same amount of cariprazine as said modified release pharmaceutical composition; when said PK profile arises from a PK experiment performed in a human fasted overnight for at least eight hours prior to dosing; wherein said PK profile is based on plasma concentrations of the total cariprazine; and wherein said pharmaceutical composition comprises cariprazine in a therapeutically effective amount.

In another preferred embodiment, the invention relates to a pharmaceutical composition comprising cariprazine, which provides a PK profile wherein Cₘₐₓ /AUC_{0-∞} is in the range of 0.05-0.20 h-1, such as in the range of 0.08-0.17 or 0.10-0.15 h-1; when said PK profile arises from a PK experiment performed in a human fasted overnight for at least eight hours prior to dosing; wherein said PK profile is based on plasma concentrations of the total cariprazine; and wherein said pharmaceutical composition comprises cariprazine in a therapeutically effective amount.

The present invention also relates to the pharmaceutical compositions as defined above for use in the treatment and/or prevention of pathological conditions which require the modulation of dopamine receptors, wherein the treatment and/or prevention comprises the administration of the pharmaceutical compositions less frequent than once daily.

In another preferred embodiment, the present invention provides a solid pharmaceutical composition as defined above for use in the treatment and/or prevention of pathological conditions which require the modulation of dopamine receptors, wherein the treatment and/or prevention comprises the administration of the pharmaceutical composition once in a 2-14 days period.

In another preferred embodiment, the present invention provides a solid pharmaceutical composition as defined above for use in the treatment and/or prevention of pathological conditions which require the modulation of dopamine receptors, wherein the treatment and/or prevention comprises the administration of the pharmaceutical composition every two days.

In another preferred embodiment, the present invention provides a solid pharmaceutical composition as defined above for use in the treatment and/or prevention of pathological conditions which require the modulation of dopamine receptors, wherein the treatment and/or prevention comprises the administration of the pharmaceutical composition every three days.

In another preferred embodiment, the present invention provides a solid pharmaceutical composition as defined above for use in the treatment and/or prevention of pathological conditions which require the modulation of dopamine receptors, wherein the treatment and/or prevention comprises the administration of the pharmaceutical composition every four days.

In another preferred embodiment, the present invention provides a solid pharmaceutical composition as defined above for use in the treatment and/or prevention of pathological conditions which require the modulation of dopamine receptors, wherein the treatment and/or prevention comprises the administration of the pharmaceutical composition every seven days.

In another preferred embodiment, the invention provides a solid pharmaceutical composition as defined above for use in the treatment and/or prevention of pathological conditions which require the modulation of dopamine receptors, wherein the treatment and/or prevention comprises the administration of the pharmaceutical composition every ten days.

In another preferred embodiment, the invention provides a solid pharmaceutical composition as defined above for use in the treatment and/or prevention of pathological conditions which require the modulation of dopamine receptors, wherein the treatment and/or prevention comprises the administration of the pharmaceutical composition every fourteen days.

In another preferred embodiment, the invention provides a solid pharmaceutical composition as defined above for use in the treatment and/or prevention of pathological conditions which require the modulation of dopamine receptors, wherein the pharmaceutical composition is divided into 2-15 monthly doses.

In another preferred embodiment, the invention provides a solid pharmaceutical composition as defined above for use in the treatment and/or prevention of pathological conditions which require the modulation of dopamine receptors, wherein the pharmaceutical composition is divided into two, three, four, five, six, seven, eight, nine, ten, eleven, twelve, thirteen, fourteen or fifteen monthly doses.

In a preferred embodiment, the invention provides a solid pharmaceutical composition as defined above for use in the treatment and/or prevention of pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, and autism.

In a more preferred embodiment, the present invention provides a solid pharmaceutical composition as defined above for use in the treatment and/or prevention of schizophrenia and/or mania.

The present invention also relates to the use of the pharmaceutical composition as defined above in the manufacture of a medicament for the treatment and/or prevention of pathological conditions which require the modulation of dopamine receptors, wherein the treatment and/or prevention comprises administration of the pharmaceutical compositions less frequent than once daily.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition once in a 2-14 days period.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition every two days.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition every three days.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition every four days.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition every seven days.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition every ten days.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises the administration of the pharmaceutical composition every fourteen days.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises administration wherein the medicament is divided into 2-15 monthly doses.

In another preferred embodiment, the present invention is directed to the use of pharmaceutical compositions as defined above in the manufacture of a medicament for the treatment and/or prevention of pathological conditions which require the modulation of dopamine receptors, wherein the treatment and/or prevention comprises administration, wherein the medicament is divided into two, three, four, five, six, seven, eight, nine, ten, eleven, twelve, thirteen, fourteen or fifteen monthly doses.

In a preferred embodiment, the present invention is directed to the use of pharmaceutical compositions as defined above in the manufacture of a medicament for the treatment and/or prevention of pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, and autism.

In a more preferred embodiment, the present invention is directed to the use of pharmaceutical compositions as defined above in the manufacture of a medicament for the treatment and/or prevention of schizophrenia and/or mania.

The present invention also relates to the process for the preparation of modified release pharmaceutical compositions as defined above in different dosage forms, wherein the compositions are obtained by conventional methods known in the art, including direct compression of the ingredients into tablets, and optionally coating them; fluid granulation and thereafter compression; and extrusion and spheronization of the ingredients and thereafter filling the obtained spheres into capsules.

In a preferred embodiment, the present invention provides a process for the preparation of modified release pharmaceutical compositions as defined above comprising the steps of
a) mixing cariprazine with suitable excipients and
b) compressing them into tablets directly.

In another preferred embodiment, the present invention provides a process for the preparation of modified release pharmaceutical compositions as defined above comprising the steps of
a) mixing cariprazine with suitable excipients in a fluid bed equipment
b) spraying the mixture with a suitable excipient dissolved in a suitable solvent

1c) drying the granules
d) covering the granules with a suitable excipient
e) mixing the granules with suitable excipients and
f) compressing the obtained mixture into tablets.

In another preferred embodiment, the present invention provides a process for the preparation of modified release pharmaceutical compositions as defined above comprising the steps of
a) mixing cariprazine with suitable excipients
b) moistening the obtained mixture
c) forming cylinder-shaped agglomerate through extrusion
d) breaking and rounding the extrudate to round spheres through spheronization
e) drying the obtained spheres, and
f) filling the spheres into suitable capsules.

In another preferred embodiment, the present invention is directed to the method of treating a patient suffering from pathological conditions which require the modulation of dopamine receptors, wherein the method comprises the administration of the pharmaceutical compositions as defined above less frequent than daily to a patient in need thereof.

In another preferred embodiment, the present invention is directed to the method of treating a patient suffering from pathological conditions which require the modulation of dopamine receptors, wherein the method comprises the administration of the pharmaceutical compositions as defined above once in 2-14 days period.

In another preferred embodiment, the present invention is directed to the method of treating a patient suffering from pathological conditions which require the modulation of dopamine receptors, wherein the method comprises the administration of the pharmaceutical compositions as defined above every two days.

In another preferred embodiment, the present invention is directed to the method of treating a patient suffering from pathological conditions which require the modulation of dopamine receptors, wherein the method comprises the administration of the pharmaceutical compositions as defined above every three days.

In another preferred embodiment, the present invention is directed to the method of treating a patient suffering from pathological conditions which require the modulation of dopamine receptors, wherein the method comprises the administration of the pharmaceutical compositions as defined above every four days.

In another preferred embodiment, the present invention is directed to the method of treating a patient suffering from pathological conditions which require the modulation of dopamine receptors, wherein the method comprises the administration of the pharmaceutical compositions as defined above every seven days.

In another preferred embodiment, the present invention is directed to the method of treating a patient suffering from pathological conditions which require the modulation of dopamine receptors, wherein the method comprises the administration of the pharmaceutical compositions as defined above every ten days.

In another preferred embodiment, the present invention is directed to the method of treating a patient suffering from pathological conditions which require the modulation of dopamine receptors, wherein the method comprises the administration of the pharmaceutical compositions as defined above every fourteen days.

In another preferred embodiment, the present invention provides the method of treating a patient suffering from pathological conditions which require the modulation of dopamine receptors, wherein the pharmaceutical composition is divided into 2-15 monthly doses.

In another preferred embodiment, the present invention provides the method of treating a patient suffering from pathological conditions which require the modulation of dopamine receptors, wherein the pharmaceutical composition is divided into two, three, four, five, six, seven, eight, nine, ten, eleven, twelve, thirteen, fourteen or fifteen monthly doses.

In a preferred embodiment, the present invention provides the method of treating a patient suffering from pathological conditions which require the modulation of dopamine receptors, such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, and autism.

In a more preferred embodiment the present invention provides the method of treating a patient suffering from schizophrenia and/or mania.

Unless otherwise indicated herein, the term “pharmaceutically acceptable salts” refers to salts obtained by reacting the main compound, functioning as a base with an inorganic or organic acid to form a salt, for example, salts of hydrochloric acid, sulfuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid. Pharmaceutically acceptable salts also include those in which the main compound functions as an acid and is reacted with an appropriate base to form, e.g., sodium, potassium, calcium, magnesium, ammonium, and choline salts. Those skilled in the art will further recognize that acid addition salts may be prepared by reaction of the compounds with the appropriate inorganic or organic acid via any of a number of known methods. Alternatively, alkali and alkaline earth metal salts can be prepared by reacting the compounds of the invention with the appropriate base via a variety of known methods.

Moreover, several acid salts can be obtained by reaction with inorganic or organic acids, namely acetates, adipates, alginates, citrates, aspartates, benzoates, benzenesulfonates, bisulfates, butyrates, camphorates, digluconates, cyclopentanepropionates, dodecyl sulfates, ethanesulfonates, glucoheptanoates, glycerophosphates, hemisulfates, heptanoates, hexanoates, fumarates, hydrobromides, hydroiodides, 2-hydroxy-ethanesulfonates, lactates, maleates, methanesulfonates, nicotinates, 2-naphthalenesulfonates, oxalates, palmoates, pectinates, persulfates, 3-phenylpropionates, picrates, pivalates, propionates, succinates, tartrates, thiocyanates, tosylates, mesylates and undecanoates.

For example, the pharmaceutically acceptable salt can be a hydrochloride salt, a hydrobromide salt or a mesylate salt.

By the term “orally deliverable”, we include the meaning suitable for oral, including peroral and intra-oral (e.g. sublingual or buccal) administration. Preferably, the compositions of the invention are designed for peroral administration to a patient, i.e. by swallowing (e.g. eating or drinking).

The term “less than daily” refers to compositions suitable for modified release dose regimens administered less frequently than once daily (OD). By dose regimens less frequent than OD, we include once every 2 days and/or every 3 days and/or every 4 days and/or every 5 days and/or every 6 days and/or every 7 days and/or every 8 days and/or every 9 days and/or every 10 days and/or every 11 days and/or every 12 days and/or every 13 days and/or every 14 days such as one dose at any time within the period of 2-14 days. In other words, by less frequent than OD, we include that the composition is divided into 2-15 monthly doses, including two, three, four, five, six, seven, eight, nine, ten, eleven, twelve, thirteen, fourteen or fifteen monthly doses.

As used herein, “bioavailability” is the oral bioavailability which is the fraction of an administered oral dose of unchanged drug that reaches the systemic circulation.

As used herein, “therapeutically effective amount” of a compound means an amount sufficient to cure, alleviate or partially arrest the clinical manifestations of a given disease and its complications in a therapeutic intervention comprising the administration of said compound. The therapeutically effective amount will vary depending on, inter alia, the disease and its severity, and on the age, weight, physical condition and responsiveness of the patient to be treated.

As used herein, “treatment” and “treating” refers to the management and care of a patient for the purpose of combating a condition, such as a disease or a disorder. The term is intended to include the full spectrum of treatments for a given condition from which the patient is suffering, such as administration of an active compound to alleviate the symptoms or complications, to delay the progression of the disease, disorder or condition, to alleviate or relieve the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications.

In the field of pharmacokinetics, the “area under the curve (AUC)” is the area under the curve (mathematically known as definite integral) in a plot of concentration of drug in blood plasma against time. Typically, the area is computed starting at the time the drug is administered and ending when the concentration in plasma is negligible. In practice, the drug concentration is measured at certain discrete points in time and the trapezoidal rule is used to estimate AUC.

As used herein, “Cₘₐₓ” is the maximum (or peak) serum concentration that a drug achieves in a specified compartment or test area of the body after the drug has been administrated and before the administration of a second dose. Short term drug side effects are most likely to occur at or near the Cₘₐₓ.

As used herein, the phrase “efficacy” is used in pharmacology and medicine to refer to both the maximum response achievable from a pharmaceutical drug in research settings, and to the capacity for sufficient therapeutic effect or beneficial change in clinical settings.

As used herein, the phrase “steady-state” refers to the situation when the rate of drug input is equal to the rate of drug elimination.

As used herein, the phrase “immediate release (IR) dosage form” of cariprazine includes the meaning that the dosage form releases substantially all of the cariprazine and its pharmaceutically acceptable salts contained therein immediately, for example within 30 minutes of administration. This definition is intended to include the compositions of cariprazine described in the introductory pages of this specification, which are currently used for treating and/or preventing pathological conditions which require the modulation of dopamine receptors.

As used herein, “modified-release tablets” are coated or uncoated tablets that contain special excipients or are prepared by special procedures, or both, designed to modify the rate, the place or the time at which the active substance(s) are released. This includes delayed-release dosage, extended-release [ER, XR, XL] dosage, and targeted-release dosage. The extended-release dosage consists of sustained-release (SR) dosage, which maintains drug release over a sustained period but not at a constant rate; and controlled-release (CR) dosage, which maintains the drug release over a sustained period at a nearly constant rate. Such modified release may also be accompanied by a higher single dose of cariprazine in the compositions of the invention compared to the currently used once daily IR formulations applied in the therapeutic dose range.

The formulations of the present invention are designed for oral administration, including, but not limited to tablets, capsules, granules, powders, microspheres, pellets, beads.

In order to achieve the modified release profile the therapeutically effective amount of cariprazine may be formulated in numerous different ways, including, but not limited to dissolution-controlled formulations, diffusion-controlled formulations, osmosis-based formulations, ion-exchange based formulations, and floating drug delivery systems.

The compositions of the invention may be dissolution-controlled formulations including, but not limited to encapsulated dissolution systems, and matrix dissolution systems. In the encapsulated dissolution system (reservoir system), the drug release can be modified by altering the thickness and the dissolution rate of the polymer membrane surrounding the drug core. In the matrix dissolution system cariprazine is homogenously distributed throughout the polymer matrix. In these systems, cariprazine may be released through diffusion mechanism as well based on the properties of the applied polymers.

The compositions of the invention may be diffusion-controlled formulations including, but not limited to reservoir systems and monolithic devices. In the reservoir systems cariprazine is surrounded by a polymer membrane, and in monolithic devices cariprazine is distributed through the polymer matrix. The reservoir systems may be nonporous membrane reservoirs or microporous membrane reservoirs; and the monolithic devices (solutions or dispersions) may be nonporous matrix or microporous matrix systems.

The compositions of the invention may be osmosis-based formulations, wherein the release rate depends on the osmotic pressure of the release medium.

The compositions of the invention may be ion-exchange based formulations, wherein the release modifying material is an ion-exchange resin, which is a water-insoluble polymeric material containing ionic groups, e.g. poly(styrene sulfonic acid).

The drug release rates can also be modified by delivering cariprazine to the stomach in a floating drug delivery system having a bulk density less than that of the gastric fluid, which system remains buoyant in the stomach for an extended period of time and increases the gastric retention time (GRT). Typically as such a system is floating on the gastric fluid, cariprazine is released slowly at the desired rate, and, after release of the drug, the residual system is emptied from the stomach resulting in better control of the fluctuations in plasma drug concentration. Floating drug delivery systems include non-effervescent and gas-generating systems.

Non-effervescent floating systems include bilayer compressed capsules, multi-layered flexible sheet-like medicament devices, hollow microspheres of acrylic resins, polystyrene floatable shells, single and multiple unit devices with floatation chambers and microporous compartments and buoyant controlled release powder formulations, or hydrogels that expand to hundreds of times their dehydrated form when immersed in water. Oral drug delivery formulations made from these gels swell rapidly in the stomach, causing medications to move more slowly from the stomach to the intestines and be absorbed more efficiently by the body. Non-effervescent floating tablets can be prepared by a combination of optimized solid dispersions of higher molecular weight fatty alcohols or fatty acid glycerides and release retarding polymers and/or swellable polymers such as xanthan gum and polyethylene oxide.

Gas-generating systems typically use effervescent components: a carbonate source and optionally an acid source. Upon coming in contact with gastric fluid, these components form CO₂ which gets entrapped in the polymer matrix typically used together with these materials. This results in the decrease of the overall density of the dosage form and thus leads to floating.

The acid source of floating dosage forms includes, but is not limited to citric acid, tartaric acid, malic acid, fumaric acid, adipic acid, succinic acid; an anhydride of said acids; an acid salt including, but not limited to sodium dihydrogen phosphate, disodium dihydrogen pyrophosphate and sodium acid sulfite and mixtures of the acids, anhydrides and acid salts.

The carbonate source includes, but is not limited to sodium bicarbonate, sodium carbonate, potassium bicarbonate, potassium carbonate, sodium sesquicarbonate, sodium glycine carbonate and mixtures thereof.

The modified drug release pattern can also be achieved by formulating a bioadhesive multiparticulate system, which is able to keep the drug substance in the small intestinal tract in order to prevent the too early elimination of the small particles.

Suitable release-modifying agents may be selected from hydrophilic and/or hydrophobic polymers and/or materials (lipid matrices and insoluble polymer matrices).

Examples of hydrophilic polymers include, but are not limited to polyethylene oxide (PEO), ethylene oxide-propylene oxide co-polymers, polyethylene-polypropylene glycol (e.g. poloxamer), carbomer, polycarbophil, chitosan, polyvinyl pyrrolidone (PVP), polyvinyl alcohol (PVA), hydroxyalkyl celluloses such as hydroxypropyl cellulose (HPC), hydroxyethyl cellulose (HEC), hydroxymethyl cellulose and hydroxypropyl methylcellulose (HPMC), carboxymethyl cellulose, sodium carboxymethyl cellulose, methylcellulose, hydroxyethyl methylcellulose, hydroxypropyl methylcellulose, polyacrylates such as carbomer, polyacrylamides, polymethacrylamides, polyphosphazines, polyoxazolidines, polyhydroxyalkylcarboxylic acids, alginic acid and its derivatives such as carrageenate alginates, ammonium alginate and sodium alginate, starch and starch derivatives, polysaccharides, carboxypolymethylene, polyethylene glycol, natural gums such as gum guar, gum acacia, gum tragacanth, karaya gum and gum xanthan, povidone, gelatin or the like.

Examples of hydrophobic polymers include, but are not limited to acrylic acid-based polymers, methacrylic acid based polymers, and acrylic acid-methacrylic acid based copolymers. As used herein, the phrase “acrylic acid-based polymers” refers to any polymer that includes one or more repeating units that include and/or are derived from acrylic acid. As used herein, the phrase “methacrylic acid-based polymers” refers to any polymer that includes one or more repeating units that include and/or are derived from methacrylic acid. Derivatives of acrylic acid and methacrylic acid include, but are not limited to, alkyl ester derivatives, alkylether ester derivatives, amide derivatives, alkyl amine derivatives, anhydride derivatives, cyanoalkyl derivatives, and amino-acid derivatives. Examples of acrylic acid-based polymers, methacrylic acid based polymers, and acrylic acid-methacrylic acid based copolymers include, but are not limited to Eudragit® L100, Eudragit® L100-55, Eudragit® L 30 D-55, Eudragit® S100, Eudragit® 4135F, Eudragit® RS, acrylic acid and methacrylic acid copolymers, methyl methacrylate polymers, methyl methacrylate copolymers, polyethoxyethyl methacrylate, polycyanoethyl methacrylate, aminoalkyl methacrylate copolymer, polyacrylic acid, polymethacrylic acid, methacrylic acid alkylamine copolymer, polymethyl methacrylate, polymethacrylic acid anhydride, polyalkylmethacrylate, polyacrylamide, and polymethacrylic acid anhydride and glycidyl methacrylate copolymers.

Hydrophilic colloids which, on contact with water, form a hydrated gel that remains intact during passage through the gastrointestinal tract are suitable matrix-forming agents for hydrophilic formulations. Examples of hydrophilic colloids include cellulose derivates, hydroxypropyl methyl cellulose, sodium carboxymethyl cellulose, alginates, xanthan gum, polyacrylic acid polymers. The rate of these agents is generally 20-80% of the composition, the actual amount depends on the drug and desired release time.

Bioadhesives and mucoadhesives are drug containing polymeric materials with the ability of adhering to biological membranes after being combined with moisture or mucus compounds. Main advantage of these drug delivery systems is their potential to prolong residence time at the site of drug absorption, and thus they can reduce the dosing frequency in modified release drug formulations. These dosage forms can also intensify the contact of their drug contents with underlying mucosal barrier, and improve the epithelial transport of drugs across mucus membranes, especially in the case if poorly absorbed drugs (Ludwig, 2005; Lehr, 2000). Synthetic polymers, such as acrylic derivatives, carbopols and polycarbophil; natural polymers, such as carageenan, pectin, acacia and alginates; and semi-synthetic polymers, like chitosan and cellulose derivatives can be used in bioadhesive formulations (Deshpande et al., 2009; Grabovac et al., 2005). Preferably cellulose derivatives, especially cellulose ethers are used in bioadhesives. More preferably nonionic cellulose ethers such as ethyl cellulose (EC), hydroxyethyl cellulose, hydoxypropyl cellulose (HPC), methyl cellulose (MC), carboxymethyl cellulose (CMC) or hydroxylpropylmethyl cellulose (HPMC) and anionic ether derivatives like sodium carboxymethyl cellulose (NaCMC) are used.

The compositions of the present invention may comprise solubilizers (e.g. polyethylene glycol, polyols, surfactants) and pH modifiers (e.g. citric acid, tartaric acid) to promote the dissolution of the active ingredient.

The compositions may also comprise one or more coating layers: a) a coating layer coated on the core, which coating layer is an inner seal coat formed of at least one coating polymer; b) a second coating layer, disposed over the inner seal coat, formed of a medicament and at least one coating polymer; and optionally c) an outer protective coating layer, disposed over the second coating layer, formed of at least one coating polymer.

The coating formulation may contain at least one coating layer material and a coating solvent, which preferably is water, which is used for processing and removed by drying. The coating layer material may be glycerol distearate; a coating layer polymer such as hydroxypropyl methylcellulose, polyvinyl alcohol (PVA), ethyl cellulose, methacrylic polymers or hydroxypropyl cellulose. The coating layer may also optionally include a plasticizer such as triacetin, diethyl phthalate, tributyl sebacate or polyethylene glycol (PEG), preferably PEG; and an anti-adherent or glidant such as talc, fumed silica or magnesium stearate, opacifying agent such as titanium dioxide. The coating layer may also include iron oxide based colorants.

In addition to the above ingredients, the compositions of the present invention may also contain suitable quantities of other pharmaceutically acceptable excipients, e.g. diluents, lubricants, binders, granulating aids, film formers, colorants, and glidants. These excipients may be used in a conventional manner, alone or in any combination.

Exemplary lubricants include, but are not limited to calcium stearate, glycerol behenate, magnesium stearate, mineral oil, polyethylene glycol, sodium stearylfumarate, stearic acid, talc, vegetable oil, zinc stearate, and combinations thereof.

Exemplary diluents include, but are not limited to microcrystalline cellulose, lactose, and starch.

Exemplary binders include, but are not limited to hydroxypropyl cellulose, hydroxypropyl methylcellulose, ethylcellulose, methylcellulose, hydroxyethyl cellulose, sugars, polyvinylpyrrolidone, polyvinyl alcohol, gum arabic powder, gelatine, pullulan, and combinations thereof.

Exemplary glidants include, but are not limited to silicon dioxide, talc, and starch.

The compositions of the present invention may be used for the therapy and/or prevention of pathological conditions which require the modulation of dopamine receptors such as psychoses (e.g. schizophrenia, schizo-affective disorders, etc.), drug abuse (e.g. alcohol, cocaine, nicotine, opioids, etc.), cognitive impairment accompanying schizophrenia (including positive symptoms, such as delusions and hallucinations, and negative symptoms, such as lack of drive and social withdrawal, and cognitive symptoms, such as problems with attention and memory), mild-to-moderate cognitive deficits, dementia, psychotic states associated with dementia, eating disorders (e.g. bulimia nervosa, etc.), attention deficit disorders, hyperactivity disorders in children, psychotic depression, mania, paranoid and delusional disorders, dyskinetic disorders (e.g. Parkinson's disease, neuroleptic induced parkinsonism, tardive dyskinesias) anxiety, sexual dysfunction, sleep disorders, emesis, aggression, autism.

Accordingly, in the light of the prior art, during the development of pharmaceutical formulations comprising active ingredients characterized by pH-dependent solubility; micro-environmental pH modulation or solubility enhancement is essential to achieve the complete dissolution of the drug.

Taking into account the characteristics of cariprazine, a person skilled in the art would expect that a complex delivery system with extra additives like pH modifiers is needed for a modified release formulation to obtain a less than daily dosage regimen keeping the same exposure as for immediate release formulations.

Therefore, we have targeted a cariprazine formulation that provides a non-immediate release (modified release) profile, as an oral depot formulation, with the potential for an effective and well tolerated less frequent non-daily dosing regimen. The modified release characteristics of the compositions can be defined in relation to their in vitro or in vivo release profiles or related values such as Cₘₐₓ and AUC, as described in more detail below.

Several formulations comprising pH modifiers and/or pharmaceutically acceptable acids and/or pharmaceutically acceptable bioadhesive polymers and/or pharmaceutically acceptable pH-dependent polymers and/or any ingredients for retention in gastrointestinal system aiming a long absorption period were tested in preclinical studies. In the pharmacokinetic phase of the development, a number of formulations was tested in plasma samples which were taken from seven dogs receiving different formulations and cariprazine concentration was analysed to compare the rate (Cₘₐₓ) and extent (AUC) of exposure, as well as Tₘₐₓ after oral administration of formulations. Two different modified release compositions and immediate release capsules, as reference samples, were tested in Phase I clinical studies and all of the modified release.

### EXAMPLES

The present invention is more specifically explained below with reference to Examples. The present invention is, however, not limited to these examples.

One group of the developed formulations (F1 and F2) is able to keep the drug substance in the acidic medium of the stomach for an extended period of time. This so-called gastroretention can be assured with floating delivery devices, which remain buoyant upon the gastric contents and thus are kept from passing through the pylorus. In order to achieve this floating behaviour, several hydrophilic swellable polymers and gas formers were tested in different molecular weight forms and quantities. Such polymers are also responsible for modified release via slow erosion and thus hindering the diffusion of the active ingredient through the swollen gel layer. Theoretically, the gastroretentive feature of the developed tablets is also advantageous because of preventing the too early elimination of the dosage form from the gastrointestinal tract (before most of the active ingredient can be released). Gastroretentive, modified-release cariprazine hydrochloride tablets are prepared by granulation of the active ingredient using alginic acid as a binder, and aqueous citric acid solution as a granulation liquid to ensure the formation of gas to promote the floating in the early phase. The sized granules in the final step are blended with release controlling matrix former agent and other excipients.

Moreover, we developed a bioadhesive multiparticulate system (F3), which is able to keep the drug substance in the upper gastrointestinal tract in order to prevent the too early elimination of the small particles. The spheres comprise a weak acid and a polyacrylic acid polymer.

Furthermore an immediate release formulation (F4) and matrix formulations (F5, F6, and F7) were developed as reference compositions.

The immediate release composition is prepared by mixing cariprazine with suitable excipients and filling the mixture into capsules.

In the matrix formulations cariprazine is embedded in an excipient that makes a non-disintegrating core called a matrix. Diffusion of (dissolved) cariprazine occurs through the core. Several different matrix formulations were developed and tested, namely matrix tablets containing a bioadhesive polymer, uncoated and intestinosolvent-coated matrix tablets containing non pH dependent polymers.

### Example 1: Floating Tablet (F1)

The F1 floating tablet is prepared by fluid granulation, wherein the cariprazine is mixed with microcrystalline cellulose and alginic acid in a fluid bed equipment; then the mixture is sprayed with an aqueous solution of citric acid. The dried granules are covered with glycerol distearate by heating the granules. In the final step the granules are mixed with the external phase (hypromellose, sodium hydrogen carbonate, colloidal anhydrous silica, magnesium stearate) and compressed into tablets using rotary tableting press equipment.

The composition contains gas forming and release modifying agents to increase the residence time in the stomach throughout the eight hours of dissolution time.

TABLE 4
Qualitative and quantitative composition of F1 floating tablet

| | Batch No. 1590 | |
|---|---|---|
| | Quantity in one dosage unit | |
| Ingredients | mg | % |
| Cariprazine hydrochloride | 19.54 | 27.91 |
| Cellulose, microcrystalline | 15.31 | 21.87 |
| Hypromellose (type 2208) | 18.00 | 25.71 |
| Glycerol distearate (type I) | 5.95 | 8.50 |
| Citric acid monohydrate | 4.20 | 6.00 |
| Sodium hydrogen carbonate | 4.00 | 5.71 |
| Alginic acid | 1.55 | 2.21 |
| Silica, colloidal anhydrous | 0.40 | 0.57 |
| Magnesium stearate (vegetable grade) | 1.05 | 1.5 |
| Total (mg/%) | 70 | 100 |

The F1 floating tablet exhibits an in vitro release profile wherein on average not more than about 15 to 35% of the total cariprazine is released within 2 hours, not more than about 50 to 70% of the total cariprazine is released within 4 hours, and not less than about 80% of the total cariprazine is released within 8 hours after placement in a standard dissolution test setting.

Dissolution method: Apparatus nr. 1 (basket); Medium—900 ml 0.001 N HCl—Run time 8 hours;

Temperature: 37±0.5° C.; Rotational speed: 50 rpm.

TABLE 5
Dissolution test results of F1 floating tablet

| | Dissolution (%)/hours | | | | | | | |
|---|---|---|---|---|---|---|---|---|
| | 1 | 2 | 3 | 4 | 5 | 6 | 7 | 8 |
| Batch No. 1590 | 26 | 40 | 51 | 61 | 70 | 77 | 86 | 92 |

### Example 2: Floating Tablet (F2)

The F2 floating tablet is prepared by fluid granulation, wherein the cariprazine is mixed with microcrystalline cellulose and alginic acid in a fluid bed equipment; and then the mixture is sprayed with an aqueous solution of citric acid. The dried granules are covered with glycerol distearate by heating the granules. In the final step the granules are mixed with the external phase (lactose monohydrate, hypromellose, sodium hydrogen carbonate, colloidal anhydrous silica, magnesium stearate) and compressed into tablets using rotary tableting press equipment.

The composition contains gas forming and release modifying agents to increase the residence time in the stomach throughout the eight hours of dissolution time.

TABLE 6
Qualitative and quantitative composition of F2 floating tablet

| | Batch No. 1591 | |
|---|---|---|
| | Quantity in one dosage unit | |
| Ingredients | mg | % |
| Cariprazine hydrochloride | 19.54 | 10.85 |
| Cellulose, microcrystalline | 50.64 | 28.13 |
| Hypromellose (type 2208) Methocel K15M | 18.00 | 10 |
| Hypromellose (type 2208) Methocel K100 | 18.00 | 10 |
| Glycerol distearate (type I) | 15.30 | 8.5 |
| Lactose monohydrate | 21.71 | 12.06 |
| Citric acid monohydrate | 10.80 | 6 |
| Sodium hydrogen carbonate | 10.29 | 5.7 |
| Alginic acid | 12.00 | 6.67 |
| Silica, colloidal anhydrous | 1.03 | 0.57 |
| Magnesium stearate (vegetable grade) | 2.70 | 1.5 |
| Total (mg/%) | 180 | 100 |

The F2 floating tablet exhibits an in vitro release profile wherein on average not more than about 20 to 40% of the total cariprazine is released within 2 hours, not more than about 45 to 65% of the total cariprazine is released within 6 hours, and not less than about 75% of the total cariprazine is released within 12 hours after placement in a standard dissolution test setting.

Dissolution method: Apparatus nr. 1 (basket); Medium—900 ml 0.001 N HCl—Run time 12 hours;

Temperature: 37±0.5° C.; Rotational speed: 50 rpm

TABLE 7
Dissolution test results of F2 floating tablet

| | Dissolution (%)/hours | | | | | | | | |
|---|---|---|---|---|---|---|---|---|---|
| | 1 | 2 | 3 | 4 | 5 | 6 | 8 | 10 | 12 |
| Batch No. 1591 | 20 | 30 | 38 | 45 | 52 | 57 | 66 | 73 | 81 |

### Example 3: Capsules Containing Bioadhesive Spheres (F3)

The F3 capsule composition is prepared by mixing cariprazine with microcrystalline cellulose and polyacrylic acid polymer in a high shear mixer; and after granulating with liquids the granulated mixture is extruded to form appropriate cylinder-shaped agglomerate, and then it is spheronized to round spheres. Before the encapsulation, the spheres are dried in fluid bed equipment; the beads are sized to the target particle size and lubricated with talc and calcium stearate. The obtained spheres are filled into hard gelatin capsules.

TABLE 8
Qualitative and quantitative composition
of F3 bioadhesive spheres for capsules

Batch No. 1626
Quantity in one dosage unit

| Ingredients | mg | % |
|---|---|---|
| Cariprazine hydrochloride | 19.62 | 10.90 |
| Cellulose, microcrystalline | 108.18 | 60.10 |
| Polyacrylic acid polymer | 9.00 | 5.00 |
| (CARBOPOL 974 P) | | |
| Lactic Acid | 21.60 | 12.00 |
| Polyethylene glycol | 21.60 | 12.00 |
| Total (mg/%) | 180 | 100 |

The F3 capsule exhibits an in vitro release profile wherein on average not more than about 55 to 65% of the total cariprazine is released within 1 hour, not more than about 74 to 86% of the total cariprazine is released within 3 hours, and not less than about 85% of the total cariprazine is released within 6 hours after placement in a standard dissolution test setting.

Dissolution method: Apparatus nr. 1 (basket); Medium—900 ml 0.001 N HCl—Run time 6 hours;

Temperature: 37±0.5° C.; Rotational speed: 50 rpm.

TABLE 9
Dissolution test results of F3 capsules

| | Dissolution (%)/hours | | | | |
|---|---|---|---|---|---|
| | 1 | 2 | 3 | 4 | 6 |
| Batch No. 1626 | 59 | 73 | 81 | 87 | 95 |

### Example 4: Immediate Release Capsule (F4)

This reference sample is prepared by mixing the ingredients and then filling the obtained mixture into hard gelatine capsule shells.

TABLE 10
Qualitative and quantitative composition of F4 reference example

| | Batch No. 1592 | |
|---|---|---|
| | Quantity in one dosage unit | |
| Ingredients | mg | % |
| Cariprazine hydrochloride | 19.54 | 19.54 |
| Starch, pregelatinised (maize) | 79.46 | 79.46 |
| Magnesium stearate (vegetable grade) | 1.00 | 1.00 |
| Total (mg/%) | 100 | 100 |

The reference capsule exhibits an in vitro release profile wherein on average more than about 85% of the total cariprazine is released within 30 minutes after placement in a standard dissolution test setting.

Dissolution method: Apparatus nr. 2 (paddle); Medium—900 ml 0.001 N HCl—Run time 30 minutes; Temperature: 37±0.5° C.; Rotational speed: 50 rpm.

TABLE 11
Dissolution test results of F4 reference capsules

Dissolution (%)/minutes

| | 5 | 10 | 15 | 30 |
|---|---|---|---|---|
| Batch No. 1592 | 46 | 98 | 100 | 100 |

### Example 5: Matrix Tablet Containing Bioadhesive Polymer (F5)

The F5 matrix tablet is prepared by mixing the ingredients and directly compressing them into tablets without using granulation or roller compaction. Cariprazine hydrochloride, dibasic calcium phosphate and colloidal anhydrous silica, are sieved together through a sieve (opening size: 1.0 mm) and the powder is blended in a double cone blender with polyacrylic acid polymer (Carbopol 974P) and it is lubricated with magnesium stearate. The lubricated powder is compressed into tablets using rotary tableting press equipment.

TABLE 12
Qualitative and quantitative composition of F5
matrix tablet containing bioadhesive polymer

Batch No. 11521
Quantity in one dosage unit

| Ingredients | mg | % |
|---|---|---|
| Cariprazine hydrochloride | 19.54 | 4.96 |
| Dibasic calcium phosphate FDC | 332.30 | 84.34 |
| Polyacrylic acid polymer | 39.40 | 10.0 |
| (CARBOPOL 974 P) | | |
| Silica, colloidal anhydrous | 0.79 | 0.2 |
| Magnesium stearate | 1.97 | 0.5 |
| Total (mg/%) | 394 | 100 |

The F5 matrix tablet exhibits an in vitro release profile wherein on average not more than about 35 to 45% of the total cariprazine is released within 2 hours, not more than about 60% of the total cariprazine is released within 4 hours, and not less than about 75% of the total cariprazine is released within 8 hours after placement in a standard dissolution test setting.

Dissolution method: Apparatus nr. 1 (basket); Medium—500 ml 0.001 N HCl—Run time 8 hours; Temperature: 37±0.5° C.; Rotational speed: 50 rpm.

TABLE 13
Dissolution test results of F5 matrix tablet

| | Dissolution (%)/hours | | | | | | | |
|---|---|---|---|---|---|---|---|---|
| | 1 | 2 | 3 | 4 | 5 | 6 | 7 | 8 |
| Batch No. 11521 | 27 | 42 | 47 | 55 | 63 | 71 | 77 | 82 |

### Example 6: Matrix Tablet Containing Non pH Dependent Polymer (F6)

The F6 matrix tablet is prepared by mixing the ingredients and compressing the mixture into tablets without using granulation or roller compaction. Cariprazine hydrochloride, microcrystalline cellulose, colloidal anhydrous silica and lactose monohydrate are sieved together through a sieve (opening size: 1.0 mm) and the powder is blended in a double cone blender with hypromellose, and it is lubricated with magnesium stearate. The lubricated powder is compressed into tablets using rotary tableting press equipment.

TABLE 14
Qualitative and quantitative composition of F6 matrix
tablet containing non pH dependent polymer

| | Batch No. 11634 | |
|---|---|---|
| | Quantity in one dosage unit | |
| Ingredients | mg | (%) |
| Cariprazine hydrochloride | 19.54 | 4.96 |
| Lactose monohydrate (Flowlac 100) | 285.81 | 72.54 |
| Microcrystalline cellulose PH 102 | 55.16 | 14.0 |
| Silica, colloidal anhydrous | 1.97 | 0.5 |
| Hypromellose (type 2208) Methocel K4M | 29.55 | 7.5 |
| Magnesium stearate | 1.97 | 0.5 |
| Total (mg/%) | 394.0 | 100 |

The F6 matrix tablet exhibits an in vitro release profile wherein on average not more than about 55 to 70% of the total cariprazine is released within 2 hours, not more than about 90% of the total cariprazine is released within 4 hours, and not less than about 95% of the total cariprazine is released within 8 hours after placement in a standard dissolution test setting.

Dissolution method: Apparatus nr. 1 (basket); Medium—500 ml 0.001 N HCl—Run time 8 hours;

Temperature: 37±0.5° C.; Rotational speed: 50 rpm.

TABLE 15
Dissolution test results of F6 matrix tablet

| | Dissolution (%)/hours | | | | | | | |
|---|---|---|---|---|---|---|---|---|
| | 1 | 2 | 3 | 4 | 5 | 6 | 7 | 8 |
| Batch No. 11634 | 48 | 67 | 86 | 89 | 95 | 96 | 96 | 97 |

### Example 7: Intestinosolvent-Coated Matrix Tablet Containing Non pH Dependent Polymer (F7)

The F7 matrix tablet is prepared by mixing the ingredients and compressing the mixture into tablets without using granulation or roller compaction. Cariprazine hydrochloride, microcrystalline cellulose, colloidal anhydrous silica and lactose monohydrate are sieved together through a sieve (opening size: 1.0 mm) and the powder is blended in a double cone blender with hypromellose, and it is lubricated with magnesium stearate. The lubricated powder is compressed into tablets using rotary tableting press equipment.

The tablets are coated with Surelease Clear E-7-19040 with a conventional coating method.

TABLE 16
Qualitative and quantitative composition of F7 intestinosolvent-
coated matrix tablet contains non pH dependent polymer

| | Batch No. 11630 | |
|---|---|---|
| | Quantity in one dosage unit | |
| Ingredients | mg | (%) |

Tablet core:

| | | |
|---|---|---|
| Cariprazine hydrochloride | 19.54 | 4.96 |
| Lactose monohydrate (Flowlac 100) | 285.81 | 72.54 |
| Microcrystalline cellulose PH 102 | 55.16 | 14.0 |
| Silica, colloidal anhydrous | 1.97 | 0.5 |
| Hypromellose (type 2208) Methocel K4M | 29.55 | 7.5 |
| Magnesium stearate | 1.97 | 0.5 |
| Total core | 394.0 | 100 |

Coat:

| | | |
|---|---|---|
| Surelease Clear E-7-19040 | 7.88 | 2.00 |
| Total film-coated tablets | 401.88 | 102 |

The F7 matrix tablet exhibits an in vitro release profile wherein on average not more than about 45 to 55% of the total cariprazine is released within 2 hours, not more than about 70% of the total cariprazine is released within 4 hours, and not less than about 90% of the total cariprazine is released within 8 hours after placement in a standard dissolution test setting.

Dissolution method: Apparatus nr. 1 (basket); Medium—500 ml 0.001 N HCl—Run time 8 hours;

Temperature: 37±0.5° C.; Rotational speed: 50 rpm

TABLE 17
Dissolution test results of F7 matrix tablet

| | Dissolution (%)/hours | | | | | | | |
|---|---|---|---|---|---|---|---|---|
| | 1 | 2 | 3 | 4 | 5 | 6 | 7 | 8 |
| Batch No. 11630 | 34 | 51 | 66 | 74 | 83 | 90 | 93 | 94 |

The purpose of the in vivo study was to provide comparative pharmacokinetic data for oral dose formulations containing cariprazine following oral tablet administration to male beagle dogs. Furthermore, the purpose of the dog PK study was to identify, from the tested prototypes, candidates to be further evaluated in a human bioavailability study.

Each group of animals received the cariprazine formulations as a single oral tablet at a target dose level of 18 mg of cariprazine. Dosing was completed without any incident.

Table 18 below shows the pharmacokinetic parameters of the formulations:

TABLE 18
Mean (CV %) PK parameters (median and min-max for Tₘₐₓ and
Fᵣₑₗ) of cariprazine after single-dose oral administration

| Tested | Cₘₐₓ | AUC₀₋ₜ | | |
|---|---|---|---|---|
| formulation | (ng/mL) | (ng/mL*h) | Fᵣₑₗ | Cₘₐₓ/AUC_{0-∞} |
| F1 | 95.8 | 758 | 0.70 | 0.13 |
| F2 | 110.6 | 886 | 0.87 | 0.12 |
| F3 | 103.3 | 755 | 0.75 | 0.14 |
| F4 | 142.3 | 1011 | NA | 0.14 |
| F5 | 109.9 | 786 | 0.84 | 0.14 |
| F6 | 110.2 | 868 | 0.79 | 0.13 |
| F7 | 100.9 | 788 | 0.81 | 0.13 |

(NA: not applicable)

Surprisingly, no statistically significant differences can be detected between the Frei (relative bioavailability, the ratio of AUC₀₋ₜ for the given formulation to that for the reference formulation) values of the different formulations, and each of the formulations decreased the value of Cₘₐₓ in comparison with the F4 reference IR capsules. Moreover, regarding the PK parameters related to exposure, no difference can be detected either.

The preclinical studies of the F1-F7 formulations showed favourable PK results in beagle dogs. However, in order to meet the requirements of human clinical trials, it was necessary to develop similar formulations for the Phase I study. Therefore, different types of formulations (PR A-E), specifically floating tablets, matrix tablets, and bioadhesive spheres were developed and tested in vitro to find the most suitable composition for the Phase I study.

### Example 8: Floating Tablet Formulation (PR A):

The PR A floating tablet is prepared by fluid granulation, wherein the cariprazine is mixed with microcrystalline cellulose and alginic acid in a fluid bed equipment; then the mixture is sprayed with an aqueous solution of citric acid. The dried granules are covered with glycerol distearate by heating the granules. In the final step the granules are mixed with the external phase (hypromellose, sodium hydrogen carbonate, colloidal anhydrous silica and magnesium stearate) and compressed into tablets using rotary tableting press equipment.

In the range of 1.5 to 24 mg of cariprazine content the different PR A formulations are qualitatively identical and in quantitative terms they are proportionally similar. All the different PR A formulations have the same nominal mass, and qualitative composition. The different dose strengths are obtained by altering the amount of cariprazine and microcrystalline cellulose.

TABLE 19
Qualitative and quantitative composition of PR A
formulations in a dose range of 1.5 mg to 9.0 mg

| | 1.5 | | | | | | | |
|---|---|---|---|---|---|---|---|---|
| | (Batch No. | | | | | | | |
| | 1B95-1B97) | | 3.0 | | 6.0 | | 9.0 | |
| Strengths | mg | % | mg | % | mg | % | mg | % |
| Cariprazine | 1.628 | 2.33 | 3.256 | 4.65 | 6.512 | 9.30 | 9.768 | 13.95 |
| hydrochloride | | | | | | | | |
| Cellulose, | 33.218 | 47.45 | 31.590 | 45.13 | 28.334 | 40.48 | 25.078 | 35.83 |
| microcrystalline | | | | | | | | |
| Hypromellose | 18.000 | 25.71 | 18.000 | 25.71 | 18.000 | 25.71 | 18.000 | 25.71 |
| (type 2208) | | | | | | | | |
| Glycerol | 5.950 | 8.50 | 5.950 | 8.50 | 5.950 | 8.50 | 5.950 | 8.50 |
| distearate (type I) | | | | | | | | |
| Citric acid | 4.200 | 6.00 | 4.200 | 6.00 | 4.200 | 6.00 | 4.200 | 6.00 |
| monohydrate | | | | | | | | |
| Sodium hydrogen | 4.000 | 5.71 | 4.000 | 5.71 | 4.000 | 5.71 | 4.000 | 5.71 |
| carbonate | | | | | | | | |
| Alginic acid | 1.554 | 2.22 | 1.554 | 2.22 | 1.554 | 2.22 | 1.554 | 2.22 |
| Silica, colloidal | 0.400 | 0.57 | 0.400 | 0.57 | 0.400 | 0.57 | 0.400 | 0.57 |
| anhydrous | | | | | | | | |
| Magnesium | 1.050 | 1.50 | 1.050 | 1.50 | 1.050 | 1.50 | 1.050 | 1.50 |
| stearate | | | | | | | | |
| (vegetable grade) | | | | | | | | |
| Total (mg/%) | 70 | 100 | 70 | 100 | 70 | 100 | 70 | 100 |

TABLE 20
Qualitative and quantitative composition of PR A
formulations in a dose range of 12 mg to 24 mg

| | | | | | | | 24 | |
|---|---|---|---|---|---|---|---|---|
| | | | | | | | (Batch No. | |
| | 12 | | 15 | | 18 | | 1B98-1B00) | |
| Strengths | mg | % | mg | % | mg | % | mg | % |
| Cariprazine | 13.024 | 18.61 | 16.28 | 23.26 | 19.535 | 27.91 | 26.047 | 37.21 |
| hydrochloride | | | | | | | | |
| Cellulose, | 21.822 | 31.18 | 18.566 | 26.52 | 15.310 | 21.87 | 8.799 | 12.57 |
| microcrystalline | | | | | | | | |
| Hypromellose | 18.00 | 25.71 | 18.00 | 25.71 | 18.00 | 25.71 | 18.00 | 25.71 |
| (type 2208) | | | | | | | | |
| Glycerol | 5.950 | 8.50 | 5.950 | 8.50 | 5.950 | 8.50 | 5.950 | 8.50 |
| distearate (type I) | | | | | | | | |
| Citric acid | 4.200 | 6.00 | 4.200 | 6.00 | 4.200 | 6.00 | 4.200 | 6.00 |
| monohydrate | | | | | | | | |
| Sodium hydrogen | 4.000 | 5.71 | 4.000 | 5.71 | 4.000 | 5.71 | 4.000 | 5.71 |
| carbonate | | | | | | | | |
| Alginic acid | 1.554 | 2.22 | 1.554 | 2.22 | 1.554 | 2.22 | 1.554 | 2.22 |
| Silica, colloidal | 0.400 | 0.57 | 0.400 | 0.57 | 0.400 | 0.57 | 0.400 | 0.57 |
| anhydrous | | | | | | | | |
| Magnesium | 1.050 | 1.50 | 1.050 | 1.50 | 1.050 | 1.50 | 1.050 | 1.50 |
| stearate | | | | | | | | |
| (vegetable grade) | | | | | | | | |
| Total (mg/%) | 70 | 100 | 70 | 100 | 70 | 100 | 70 | 100 |

The PR A floating tablet exhibits an in vitro release profile wherein on average not more than about 20 to 40% of the total cariprazine is released within 2 hours, not more than about 48 to 75% of the total cariprazine is released within 4 hours, and not more than about 80% of the total cariprazine is released within 8 hours after placement in a standard dissolution test setting.

Dissolution method: Apparatus nr. 2 (paddle); Medium—900 ml 0.001 N HCl solution—Run time 12 hours; Temperature: 37±0.5° C.; Rotational speed: 50 rpm.

TABLE 21
Dissolution test results of different PR A formulations

| | | |
|---|---|---|
| | 1.5 mg | 24 mg |
| Time | release (%) | release (%) |

| (hour) | 1B95 | 1B96 | 1B97 | 1B98 | 1B99 | 1B00 |
|---|---|---|---|---|---|---|
| 2 | 33 | 34 | 32 | 26 | 25 | 25 |
| 4 | 53 | 50 | 47 | 43 | 41 | 42 |
| 6 | 73 | 67 | 64 | 57 | 56 | 56 |
| 8 | 90 | 82 | 81 | 70 | 68 | 69 |
| 10 | 100 | 93 | 92 | 80 | 79 | 79 |
| 12 | 105 | 100 | 100 | 90 | 88 | 87 |

The “alcohol-induced dose dumping” was examined as well, in media containing different amount of ethanol and it was found that the dissolution of the drug was not modified. Therefore, this composition provides a safe use for patients who consume hydro-alcoholic liquids during the treatment period.

TABLE 22
Dose dumping dissolution test results of PR A formulation

| | |
|---|---|
| | 1.5 mg |
| Time | Batch No. 1B97 |
| (min) | release (%) |

| | | | | |
|---|---|---|---|---|
| Medium | 1 | 2 | 3 | 4 |
| 15 | 3 | 8 | 9 | 10 |
| 30 | 12 | 15 | 15 | 17 |
| 45 | 17 | 19 | 20 | 21 |
| 60 | 20 | 23 | 23 | 25 |
| 75 | 22 | 27 | 37 | 28 |
| 90 | 24 | 30 | 31 | 32 |
| 105 | 25 | 34 | 34 | 35 |
| 120 | 27 | 36 | 37 | 38 |

Dissolution method: Apparatus nr. 1 (basket); Medium 1-500 ml 0.1 N HCl solution—Run time 2 hours; Medium 2-500 ml Ethanol/HCl 0.1 N (5%)—Run time 2 hours; Medium 3-500 ml Ethanol/HCl 0.1 N (20%)—Run time 2 hours; Medium 4-500 ml Ethanol/HCl 0.1 N (40%)—Run time 2 hours; Temperature: 37±0.5° C.; Rotational speed: 50 rpm.

### Example 9: Matrix Tablet Containing Non pH Dependent Polymer (PR B)

The PR B matrix tablet is prepared by mixing the ingredients and compressing the mixture into tablets without using granulation or roller compaction. Cariprazine hydrochloride, microcrystalline cellulose, colloidal anhydrous silica, and lactose monohydrate are sieved together through a sieve (opening size: 1.0 mm) and the powder is blended in a double cone blender with hypromellose, and it is lubricated with magnesium stearate. The lubricated powder is compressed into tablets using rotary tableting press equipment.

In the range of 1.5 to 24 mg of cariprazine content the different PR B formulations are qualitatively identical and in quantitative terms they are proportionally similar. All the different PR B formulations have the same nominal mass and qualitative composition. The different dose strengths are obtained by altering the amount of cariprazine and lactose monohydrate.

TABLE 23
Qualitative and quantitative composition of PR B
formulations in a dose range of 1.5 mg to 9.0 mg

| | 1.5 | | | | | | | |
|---|---|---|---|---|---|---|---|---|
| | (Batch No. | | | | | | | |
| | 11C58-11C60) | | 3.0 | | 6.0 | | 9.0 | |
| Ingredients | mg | % | mg | % | mg | % | mg | % |
| Cariprazine | 1.628 | 1.3 | 3.256 | 2.6 | 6.512 | 5.2 | 9.768 | 7.8 |
| hydrochloride | | | | | | | | |
| Lactose | 54.622 | 43.7 | 52.994 | 42.4 | 49.738 | 39.8 | 46.482 | 37.2 |
| monohydrate | | | | | | | | |
| Cellulose | 17.5 | 14.0 | 17.5 | 14.0 | 17.5 | 14.0 | 17.5 | 14.0 |
| microcrystalline | | | | | | | | |
| type 102 | | | | | | | | |
| Hypromellose | 50.0 | 40.0 | 50.0 | 40.0 | 50.0 | 40.0 | 50.0 | 40.0 |
| type 2208 | | | | | | | | |
| Silica, colloidal | 0.625 | 0.5 | 0.625 | 0.5 | 0.625 | 0.5 | 0.625 | 0.5 |
| anhydrous | | | | | | | | |
| Magnesium | 0.625 | 0.5 | 0.625 | 0.5 | 0.625 | 0.5 | 0.625 | 0.5 |
| stearate | | | | | | | | |
| Total (mg/%) | 125.0 | 100.0 | 125.0 | 100.0 | 125.0 | 100.0 | 125.0 | 100.0 |

TABLE 24
Qualitative and quantitative composition of PR B
formulations in a dose range of 12 mg to 24 mg

| | | | | | | | 24 | |
|---|---|---|---|---|---|---|---|---|
| | | | | | | | (Batch No. | |
| | 12 | | 15 | | 18 | | 11C61-11C63) | |
| Ingredients | mg | % | mg | % | mg | % | mg | % |
| Cariprazine | 13.024 | 10.4 | 16.280 | 13.0 | 19.537 | 15.6 | 26.047 | 9.5 |
| hydrochloride | | | | | | | | |
| Lactose | 43.226 | 34.6 | 39.970 | 32.0 | 36.713 | 29.4 | 159.578 | 58.0 |
| monohydrate | | | | | | | | |
| Cellulose | 17.5 | 14.0 | 17.5 | 14.0 | 17.5 | 14.0 | 38.5 | 14.0 |
| microcrystalline | | | | | | | | |
| type 102 | | | | | | | | |
| Hypromellose | 50 | 40.0 | 50.0 | 40.0 | 50.0 | 40.0 | 48.125 | 17.5 |
| type 2208 | | | | | | | | |
| Silica, colloidal | 0.625 | 0.5 | 0.625 | 0.5 | 0.625 | 0.5 | 1.375 | 0.5 |
| anhydrous | | | | | | | | |
| Magnesium | 0.625 | 0.5 | 0.625 | 0.5 | 0.625 | 0.5 | 1.375 | 0.5 |
| stearate | | | | | | | | |
| Total (mg/%) | 125.0 | 100.0 | 125.0 | 100.0 | 125.0 | 100.0 | 275.0 | 100.0 |

The PR B composition exhibits an in vitro release profile wherein on average not more than about 15 to 35% of the total cariprazine is released within 1 hour, not more than about 40 to 60% of the total cariprazine is released within 3 hours, and not more than about 75% of the total cariprazine is released within 12 hours after placement in a standard dissolution test setting.

Dissolution method: Apparatus nr. 1 (basket); Medium 1-500 ml 0.1 N HCl solution—Run time 2 hours; Medium 2-500 ml Acetate buffer with pH=5.0—Run time 10 hours; Temperature: 37±0.5° C.; Rotational speed: 50 rpm.

TABLE 25
Dissolution test results of different PR B formulations

| | 1.5 mg | | | 24 mg | | |
|---|---|---|---|---|---|---|
| Time | release (%) | | | release (%) | | |
| (hour) | 11C58 | 11C59 | 11C60 | 11C61 | 11C62 | 11C63 |
| 1 | 21 | 23 | 23 | 24 | 23 | 26 |
| 1.5 | 27 | 30 | 30 | 31 | 31 | 32 |
| 2 | 33 | 37 | 37 | 37 | 37 | 38 |
| 3 | 45 | 49 | 48 | 46 | 47 | 48 |
| 4 | 54 | 58 | 57 | 50 | 50 | 51 |
| 5 | 59 | 64 | 63 | 53 | 54 | 55 |
| 6 | 63 | 68 | 67 | 56 | 58 | 58 |
| 7 | 66 | 71 | 70 | 60 | 61 | 62 |
| 8 | 68 | 73 | 73 | 64 | 64 | 65 |
| 10 | 73 | 78 | 77 | 71 | 70 | 71 |
| 12 | 79 | 82 | 81 | 79 | 78 | 81 |

The “alcohol-induced dose dumping” was examined as well, in a media containing different amounts of ethanol and it was found that the dissolution of the drug was not modified. Therefore, this composition provides a safe use for patients who consume hydro-alcoholic liquids during the treatment period.

TABLE 26
Dose dumping dissolution test results
of different PR B formulations

| | |
|---|---|
| 1.5 mg | 24 mg |
| Batch No. 11C58 | Batch No. 11C60 |
| release (%) | release (%) |

| | |
|---|---|
| Time | Medium |

| (min) | 1 | 2 | 3 | 4 | 1 | 2 | 3 | 4 |
|---|---|---|---|---|---|---|---|---|
| 15 | 5 | 7 | 7 | 6 | 9 | 9 | 7 | 7 |
| 30 | 12 | 13 | 11 | 12 | 13 | 14 | 12 | 12 |
| 45 | 16 | 17 | 16 | 16 | 16 | 18 | 16 | 17 |
| 60 | 20 | 22 | 20 | 20 | 20 | 21 | 20 | 21 |
| 75 | 24 | 26 | 23 | 23 | 22 | 24 | 23 | 26 |
| 90 | 28 | 29 | 26 | 27 | 25 | 26 | 26 | 28 |
| 105 | 31 | 33 | 29 | 30 | 28 | 29 | 28 | 31 |
| 120 | 34 | 36 | 32 | 32 | 30 | 31 | 31 | 33 |

Dissolution method: Apparatus nr. 1 (basket); Medium 1-500 ml 0.1 N HCl solution—Run time 2 hours; Medium 2-500 ml Ethanol/HCl 0.1 N (5%)—Run time 2 hours; Medium 3-500 ml Ethanol/HCl 0.1 N (20%)—Run time 2 hours; Medium 4-500 ml Ethanol/HCl 0.1 N (40%)—Run time 2 hours; Temperature: 37±0.5° C.; Rotational speed: 50 rpm.

### Example 10: Matrix Tablet Containing Non pH Dependent Polymer (PR C)

The PR C matrix tablet is prepared by mixing the ingredients and compressing the mixture into tablets without using granulation or roller compaction. Cariprazine hydrochloride, microcrystalline cellulose and/or lactose monohydrate and/or calcium hydrogen phosphate, and colloidal anhydrous silica are sieved together through a sieve (opening size: 1.0 mm) and the powder is blended in a double cone blender with hypromellose or ethylcellulose, and it is lubricated with magnesium stearate. The lubricated powder is compressed into tablets using rotary tableting press equipment. The obtained tablets are optionally coated with Opadry and Acryl EZE with any conventional method.

TABLE 27
Qualitative and quantitative composition of the PR C formulations

| | Batch No. 11301 | | Batch No. 11302 | | Batch No. 11406 | |
|---|---|---|---|---|---|---|
| Ingredients | mg | % | mg | % | mg | % |

Tablet core:

| | | | | | | |
|---|---|---|---|---|---|---|
| Cariprazine hydrochloride | 1.63 | 1.30 | 1.63 | 1.30 | 1.63 | 1.30 |
| Lactose monohydrate | 73.37 | 58.70 | — | — | — | — |
| Calcium hydrogen | — | — | 73.37 | 58.70 | 97.5 | 78.0 |
| phosphate | | | | | | |
| Cellulose microcrystalline | 7.5 | 6.00 | 7.5 | 6.00 | — | — |
| type 102 | | | | | | |
| Hypromellose type 2208 | 41.25 | 33.00 | 41.25 | 33.00 | — | — |
| Ethylcellulose (Aquaion | — | — | — | — | 25.0 | 20.0 |
| T10) | | | | | | |
| Silica, colloidal | 0.63 | 0.5 | 0.63 | 0.5 | 0.25 | 0.20 |
| anhydrous | | | | | | |
| Magnesium stearate | 0.63 | 0.5 | 0.63 | 0.5 | 0.62 | 0.50 |
| Total core | 125 | 100 | 125 | 100 | 125 | 100 |
| (mg/%) | | | | | | |

Coating (optional)

| | | | | | | |
|---|---|---|---|---|---|---|
| Opadry YS-1-7027 | 2.5 | 2.0 | — | — | — | — |
| Acryl EZE 93F19255 | 12.75 | 10.0 | — | — | — | — |
| Total (mg/%) | 140.25 | 112.4 | — | — | — | — |

The PR C formulations exhibit an in vitro release profile wherein on average not more than about 15 to 35% of the total cariprazine is released within 1 hour, not more than about 40 to 70% of the total cariprazine is released within 3 hours, and not more than about 75% of the total cariprazine is released within 7 hours after placement in a standard dissolution test setting.

Dissolution method (11301, 11302): Apparatus nr. 1 (basket); Medium 1-500 ml 0.1 N HCl solution—Run time 2 hours; Medium 2-500 ml Acetate buffer with pH=5.0—Run time 4 hours; Temperature: 37±0.5° C.; Rotational speed: 50 rpm.

Dissolution method (11406): Apparatus nr. 1 (basket); Medium 1-500 ml 0.1 N HCl solution—Run time 2 hours; Medium 2-500 ml Acetate buffer with pH=5.5—Run time 12 hours;

Temperature: 37±0.5° C.; Rotational speed: 50 rpm.

TABLE 28
Dissolution test results of PR C formulations

| Time | Batch No. 11301 | Batch No. 11302 | Batch No. 11406 |
|---|---|---|---|
| (min) | release (%) | release (%) | release (%) |
| Medium | 1 | 1 | 1 |
| 60 | 34 | 26 | 31 |
| 90 | 44 | 38 | 46 |
| 120 | 54 | 47 | 54 |
| Medium | 2 | 2 | 3 |
| 15 | — | — | 31 |
| 30 | — | — | 46 |
| 45 | — | — | 54 |
| 60 | 66 | 63 | 66 |
| 90 | — | — | 75 |
| 120 | 77 | 76 | 77 |
| 180 | 86 | 86 | 79 |
| 240 | 93 | 92 | 80 |

### Example 11: Capsules Containing Bioadhesive Spheres (PR D)

The PR D capsule is prepared by mixing cariprazine with microcrystalline cellulose and polyacrylic acid polymer in a high shear mixer; and after granulating with liquids the granulated mixture is extruded to form appropriate cylinder-shaped agglomerate, and then it is spheronized to round spheres. Before the encapsulation, the beads are dried in fluid bed equipment, then the beads are sized to the target particle size and lubricated with talc and calcium stearate. The obtained spheres are filled into hard gelatin capsules.

TABLE 29
Qualitative and quantitative composition of the PR D formulations

| | Quantity in one dosage unit | | | |
|---|---|---|---|---|
| | 1.5 mg | | 24 mg | |
| | (Batch No. 2308, | | (Batch No. 2312, | |
| | 2310, 2311) | | 2313, 2315) | |
| | mg/ | | mg/ | |
| Ingredients | capsules | (%) | capsules | (%) |
| Cariprazine hydrochloride | 1.628 | 4.28 | 26.048 | 4.28 |
| Cellulose, microcrystalline | 26.302 | 69.22 | 420.832 | 69.22 |
| type 101 | | | | |
| Polyacrylic acid polymer | 2.850 | 7.50 | 45.600 | 7.50 |
| (CARBOPOL 974 P) | | | | |
| Calcium chloride | 1.140 | 3.00 | 18.240 | 3.00 |
| Caprylocaproyl | 3.800 | 10.00 | 60.800 | 10.00 |
| polyoxylglycerides | | | | |
| Polyethylene glycol | 1.900 | 5.00 | 30.400 | 5.00 |
| Talc | 0.190 | 0.50 | 3.040 | 0.50 |
| Calcium stearate | 0.190 | 0.50 | 3.040 | 0.50 |
| Total (%/mg) | 38 | 100 | 608 | 100 |

The PR D formulations exhibit an in vitro release profile wherein on average not more than about 15 to 45% of the total cariprazine is released within 1 hour, not more than about 48 to 80% of the total cariprazine is released within 3 hours, and not less than about 80% of the total cariprazine is released within 8 hours after placement in a standard dissolution test setting.

Dissolution method: Apparatus nr. 1 (basket); Medium -900 ml Acetate buffer with pH=5.0—Run time 8 hours; Temperature: 37±0.5° C.; Rotational speed: 50 rpm.

TABLE 30
Dissolution test results of different PR D formulations

| | | |
|---|---|---|
| | 1.5 mg | 24 mg |
| Time | release (%) | release (%) |

| (hour) | 2308 | 2310 | 2311 | 2312 | 2313 | 2315 |
|---|---|---|---|---|---|---|
| 0 | 0 | 0 | 0 | 0 | 0 | 0 |
| 1 | 26 | 23 | 23 | 38 | 37 | 38 |
| 2 | 48 | 43 | 44 | 60 | 60 | 61 |
| 3 | 64 | 59 | 61 | 74 | 76 | 76 |
| 4 | 77 | 72 | 74 | 85 | 87 | 87 |
| 6 | 94 | 90 | 91 | 96 | 99 | 98 |
| 8 | 101 | 99 | 98 | 100 | 101 | 102 |

### Example 12: Capsules Containing Bioadhesive Spheres (PR E)

The PR E formulations are prepared similarly to the PR D capsules (Example 11). The difference between the compositions, is that the PR E formulations do not contain any electrolytes, such as CaCl₂, in order to achieve a better elasticity of the spheres,

TABLE 31
Qualitative and quantitative composition
of different PR E compositions

| | Quantity in one dosage unit | | | |
|---|---|---|---|---|
| | 1.5 mg | | 24 mg | |
| | (Batch No. | | (Batch No. | |
| | 1C73, 1C74) | | 1C76-1C78) | |
| | mg/ | | mg/ | |
| Ingredients | capsules | (%) | capsules | (%) |
| Cariprazine hydrochloride | 1.628 | 10.85 | 26.048 | 10.85 |
| Cellulose, microcrystalline | 9.997 | 66.65 | 159.960 | 66.65 |
| type 101 | | | | |
| Polyacrylic acid polymer | 1.125 | 7.5 | 18.000 | 7.5 |
| (CARBOPOL 974 P) | | | | |
| Caprylocaproyl | 1.500 | 10.0 | 24.000 | 10.0 |
| polyoxylglycerides | | | | |
| Polyethylene glycol | 0.750 | 5 | 12.000 | 5 |
| Total (%/mg) | 15 | 100 | 240 | 100 |

The PR E formulations exhibit an in vitro release profile wherein on average not more than about 15 to 45% of the total cariprazine is released within 2 hours, not more than about 48 to 80% of the total cariprazine is released within 10 hours, and not less than about 80% of the total cariprazine is released within 16 hours after placement in a standard dissolution test.

Dissolution method: Apparatus nr. 1 (basket); Medium—900 ml Acetate buffer with pH=5.0—Run time 8 hours; Temperature: 37±0.5° C.; Rotational speed: 50 rpm.

TABLE 32
Dissolution test results of different PR E formulations

| | | |
|---|---|---|
| | 1.5 mg | 24 mg |
| Time | release (%) | release (%) |

| (hour) | 1C73 | 1C74 | 1C76 | 1C77 | 1C78 |
|---|---|---|---|---|---|
| 2 | 32 | 24 | 29 | 30 | 28 |
| 4 | 45 | 38 | 43 | 45 | 43 |
| 6 | 56 | 51 | 53 | 56 | 54 |
| 8 | 67 | 61 | 62 | 66 | 64 |
| 10 | 77 | 72 | 71 | 75 | 73 |
| 12 | 85 | 81 | 79 | 82 | 82 |
| 14 | 91 | 88 | 86 | 90 | 89 |
| 16 | 96 | 93 | 92 | 96 | 95 |

The ratio of liquid to solid material together with the size, particle size distribution and smoothness of the extruder holes surface significantly determines the quality of the extrudates. The final drying ensures the pellet hardness.

This approach is known to reduce the viscosity of polyacrylic acids by disturbing the interactions between carboxylate groups on adjacent polymer molecules, thereby decreasing their bioadhesive properties, but it significantly decreases the elasticity of the extrudates which is critical in the spheronization step. The amount of water, extrusion speed, spheronization speed and time need to be optimized in order to obtain the highest yields and sphericity. In the presence of electrolytes (e.g. calcium chloride), the processing is easier, but the electrolyte has a negative effect on the bioadhesion and drug release. Furthermore, the use of electrolytes did not solve the problem completely, as the shape of the beads is not spherical and the stickiness causes agglomeration during the process.

Moreover, a non-ionic surfactant was tried to ensure the complete dissolution of cariprazine in the upper intestinal tract during the long release time. It has surprisingly been found, that using liquids for improving the solubility completely solves the sticking problem and spherical beads can be gained. The compositions of the present invention comprise solubility enhancer solvents, selected from a group consisting of caprylocaproyl macrogolglycerides, 1,2,3-propanetriol, lactic acid, lauroyl polyoxylglycerides, polyoxylglycerides, polyoxyethylene glycol, 2-hydroxypropanol.

However, the process for the preparation of bioadhesive spheres is complicated and economically not advantageous, thus it is not the most preferred embodiment of the present invention.

### Example 13: A Single-Dose Phase I Study

Based on the preclinical PK results and economic considerations two different types of the formulations were selected (PR A and PR B) for clinical investigations to compare them as they have the most suitable AUC (exposure) results along with low Cₘₐₓ values.

A single-dose Phase I study was designed to evaluate the pharmacokinetic profiles of the two above mentioned modified release formulations compared to the immediate release formulation in healthy men.

The summary of the descriptive statistics of main Cariprazine PK parameters following single dose administration of the IR, PR A and PR B formulations in healthy male volunteers is given in Table 33 below (Cₘₐₓ: Maximum observed plasma concentration; Tₘₐₓ: Time of observed Cₘₐₓ; AUC₀₋ₜ: Area under the plasma concentration—time curve from time zero to the last quantifiable concentration; Tₗₐₛₜ: Time of last quantifiable concentration; AUC %_{Extra}: Area under the plasma concentration—time curve from time zero to infinity (extrapolated); AUC %_{Extra}: Percentage of extrapolated area to AUC_{0-∞}; MRT_{0-∞}: Mean residence time from time zero to infinity (extrapolated); t^{1/2}: Apparent terminal half-life; CL/F: Apparent oral clearance; V_{z}/F: Apparent volume of distribution):

TABLE 33
Descriptive statistics of pharmacokinetic parameters of Total Cariprazine after single oral administration
of the IR, PR A or PR B formulations at the dose of 1.5 mg to healthy male volunteers

| | | | | | | | | | |
|---|---|---|---|---|---|---|---|---|---|
| Cₘₐₓ | Tₘₐₓ | AUC₀₋ₜ | Tₗₐₛₜ | AUC_{0-∞} | AUC %_{Extra} | MRT_{0-∞} | t_{1/2} | CL/F | V_{z}/F |
| (nmol/L) | (h) | (h*nmol/L) | (h) | (h*nmol/L) | (%) | (h) | (h) | (Uh) | (L) |

Total cariprazine
IR treatment

| | | | | | | | | | | |
|---|---|---|---|---|---|---|---|---|---|---|
| N | 36 | 36 | 36 | 36 | 36 | 36 | 36 | 36 | 36 | 36 |
| Mean | 2.834 | 4 | 240 | 653 | 329 | 26.5 | 492 | 386 | 11.42 | 6114 |
| SD | 0.902 | 2 | 60 | 78 | 84 | 7.8 | 150 | 115 | 3.22 | 1988 |
| CV % | 31.8 | 46.9 | 25.1 | 11.9 | 25.5 | 29.6 | 30.4 | 29.7 | 28.2 | 32.5 |
| Min | 1.250 | 2 | 128 | 336 | 166 | 10.7 | 191 | 143 | 7.32 | 3206 |
| Median | 2.713 | 3 | 238 | 672 | 324 | 26.9 | 497 | 402 | 10.82 | 5906 |
| Max | 5.019 | 9 | 396 | 672 | 479 | 44.8 | 864 | 654 | 21.08 | 14118 |

PRA treatment

| | | | | | | | | | | |
|---|---|---|---|---|---|---|---|---|---|---|
| N | 37 | 37 | 37 | 37 | 37 | 37 | 37 | 37 | 37 | 37 |
| Mean | 1.027 | 33 | 205 | 667 | 286 | 28.1 | 530 | 393 | 12.98 | 7152 |
| SD | 0.428 | 14 | 51 | 28 | 70 | 7.8 | 134 | 106 | 3.17 | 1819 |
| CV % | 41.6 | 43.0 | 25.1 | 4.1 | 24.4 | 27.7 | 25.3 | 26.9 | 24.4 | 25.4 |
| Min | 0.505 | 5 | 136 | 504 | 177 | 13.2 | 321 | 243 | 7.68 | 3677 |
| Median | 0.857 | 36 | 194 | 672 | 275 | 27.5 | 513 | 375 | 12.77 | 7333 |
| Max | 2.522 | 72 | 359 | 672 | 457 | 46.1 | 912 | 703 | 19.82 | 10554 |

PRB treatment

| | | | | | | | | | | |
|---|---|---|---|---|---|---|---|---|---|---|
| N | 37 | 37 | 37 | 37 | 37 | 37 | 37 | 37 | 37 | 37 |
| Mean | 0.950 | 34 | 196 | 672 | 284 | 29.3 | 567 | 428 | 13.47 | 7915 |
| SD | 0.272 | 11 | 50 | 0 | 86 | 10.4 | 204 | 151 | 4.12 | 2489 |
| CV % | 28.6 | 32.3 | 25.6 | 0.0 | 30.2 | 35.6 | 35.9 | 35.3 | 30.6 | 31.5 |
| Min | 0.436 | 5 | 108 | 672 | 130 | 13.8 | 318 | 251 | 6.35 | 4501 |
| Median | 0.934 | 36 | 185 | 672 | 272 | 27.9 | 519 | 408 | 12.90 | 7443 |
| Max | 1.628 | 48 | 308 | 672 | 553 | 53.8 | 1144 | 849 | 27.03 | 14866 |

Overall the two prolonged release formulations showed total cariprazine exposures (AUC_{0-∞}) comparable with those of the IR formulation, while delaying and decreasing the maximal plasma concentration (Cₘₐₓ). It has surprisingly been found that the two prolonged release formulations: floating tablets positioned in acidic environment and simple matrix tablets, were very similar to each other.

Median of Tₘₐₓ values of total cariprazine for the IR formulation was 3 hours, while for the prolonged release formulations (PR A and PR B) the median Tₘₐₓ values were delayed to 36 hours. Mean (±SD) Cₘₐₓ for total cariprazine was 2.834 (±0.902) nmol/L for the IR formulation and decreased to 1.027 (±0.428) and 0.950 (±0.272) nmol/L for the prolonged release formulations PR A and PR B, respectively. Mean (±SD) AUC_{0-∞} value for total cariprazine was 329 (±84) h*nmol/L for the IR formulation and 286 (±70) and 284 (±86) h*nmol/L for the PR formulations PR A and PR B, respectively.

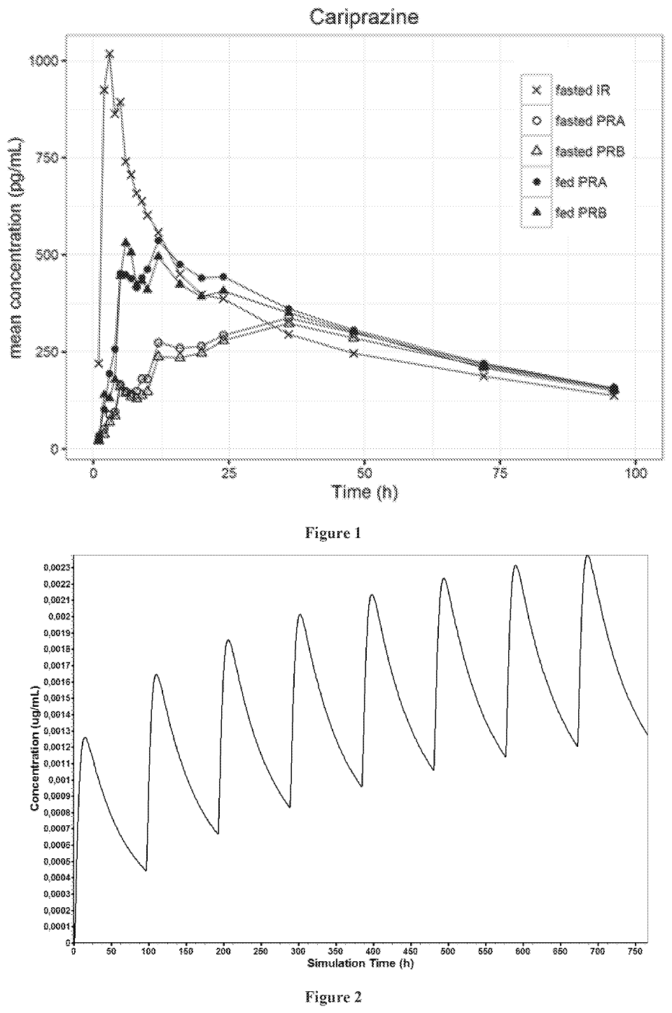

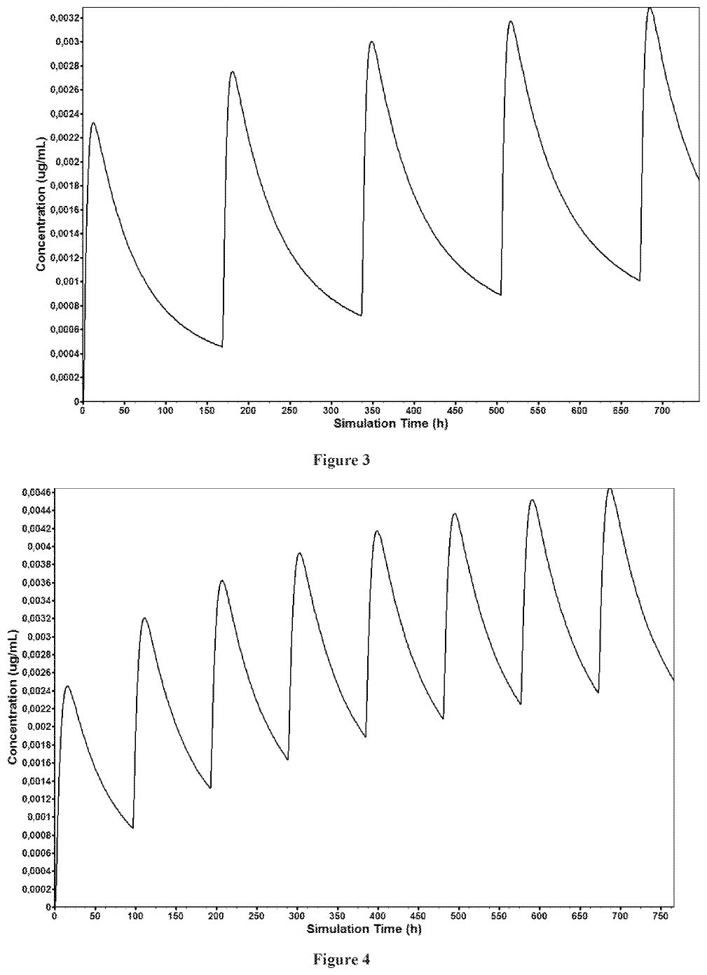

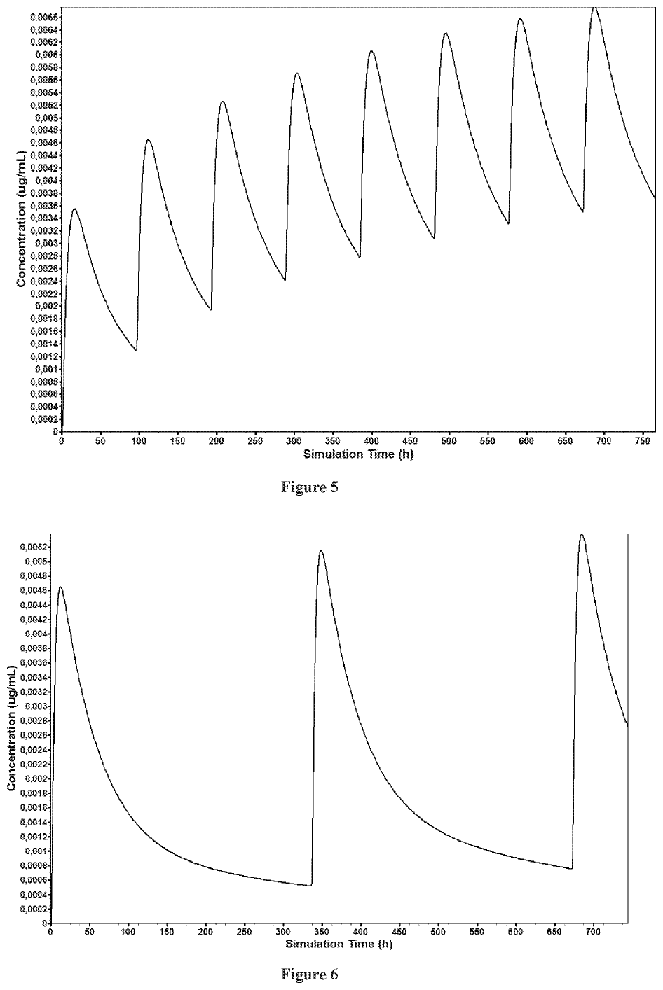

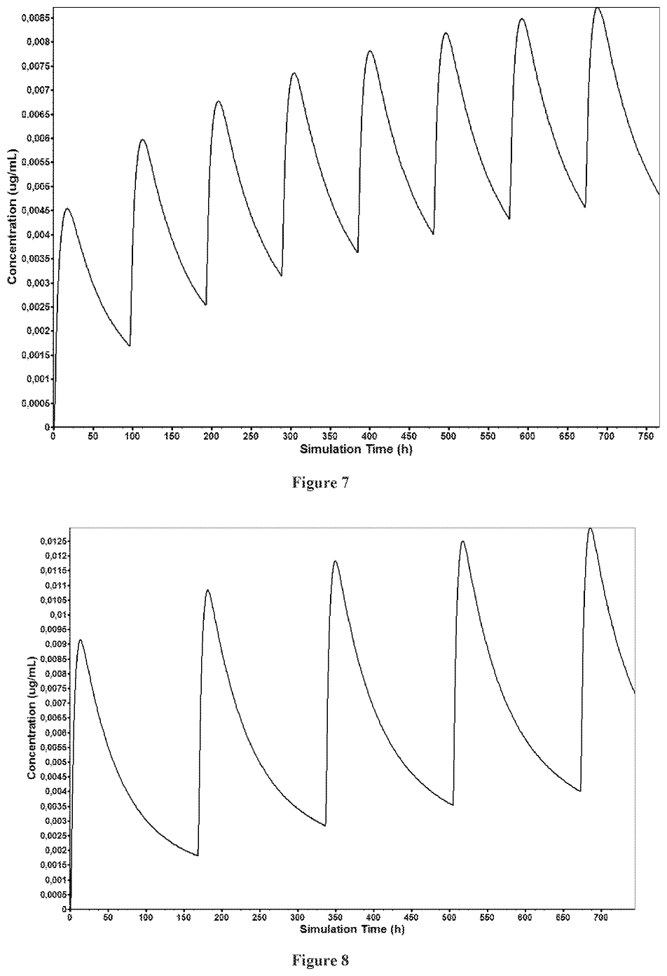

Mean plasma concentration—time profiles of total cariprazine after single oral administration of the IR, PR A and PR B formulations at the dose of 1.5 mg to healthy male volunteers are illustrated in FIG. 1.

The results showed that single doses of cariprazine 1.5 mg administered as PR A tablet and PR B tablet gave similar PK in healthy volunteers. The two PR tablets resulted in systemic exposure (AUC_{0-∞} or AUC₀₋ₜ) to cariprazine that were comparable to those of the IR capsule under fasted conditions, while Cₘₐₓ of each analyte was lower, and Tₘₐₓ was later, than for the IR capsule. Both PR formulations showed comparable systemic exposures (AUC_{0-∞}) under fasted and fed conditions. In general, the two modified release formulations had similar food effect.

In the light of the prior art, the skilled person would expect that both the polymer compounds and the acidifier and/or agents ensuring gastric positioning (such as carbonate source and/or bioadhesive compounds) are essential components for the development of a pharmaceutical formulation comprising cariprazine. Contrary to this, it has unexpectedly been found, that still the most simple matrix tablet formulation without using any special additives is able to provide appropriate extended release system having pH-independent bioavailability for a cariprazine hydrochloride composition. The matrix tablet form without any pH modifier and/or gas forming agent and/or bioadhesive material showed the same characteristics as the more sophisticated complex systems including a number of special additives; namely the AUC value did not decrease and Cₘₐₓ value did not increase.

These were surprising results, taking into consideration that the most important PK parameters were the same for the two different modified release formulations. It was concluded that the simple PR B composition without any additive agent and simple manufacturing process showed the desired characteristics of a modified release cariprazine formulation and actually the PR B composition challenges the more complicated PR A composition. Since the PR B tablet accomplishes the objective of the development and performs the tasks well, the use of a more complex delivery system e.g. a floating tablet or bioadhesive spheres is not required for a perfect operation of the new dosage regimen including cariprazine hydrochloride or its pharmaceutically acceptable salts.

Consequently, the development brought a real surprise as it is proved that the development of complicated gastroretentive floating systems, and the use of bioadhesive polymers, pH-dependent excipients and pH modifying agents in the different delivery systems is not required, because surprisingly the PR B tablet meets all the expectations. At the same time, utilization of more complex delivery systems is generally susceptible in the required stability tests and technologically more demanding (due to the use of special additives, difficult manufacturing and equipment systems) while they cannot provide any significant benefit.

From clinical point of view the less frequent, less than daily administration of an oral formulation is advantageous especially on the long term such as diseases of central nervous system including schizophrenia. To reach this goal a modified release formulation of cariprazine was required with almost the same systemic exposure of the IR capsule and not higher Cₘₐₓ than the Cₘₐₓ of the IR capsule. Having all of the results of analytical tests, preclinical and clinical studies of several modified release systems it was concluded that the desired features of a modified release cariprazine formulation with suitable systemic exposure for less than daily administration were well achieved with a simple matrix composition without any special agents. We found these results as the most unexpected and surprising results.

### Example 14: Pharmacokinetic Modelling

Immediate release (IR) formulations of cariprazine are typically administered at low doses (e.g., 1.5-6 mg/day) and progressively administered at increasing frequency and dose over time to reach a steady-state serum concentration that is therapeutically effective. According to the FDA approved label, an immediate release (IR) formulation of cariprazine, is first administered to subjects at a dose of 1.5 mg per day. Using a modified release formulation comprising higher dose of cariprazine, a therapeutically effective steady-state concentration may be achieved substantially sooner, without using a dose escalating regimen, but this is not yet acceptable in this stage of the development. Accordingly, the Cₘₐₓ of the modified release formulation is reduced compared to the immediate release formulation even though the dose administered is larger than for the immediate release formulation. In order to determine the most suitable formulation, the pharmacokinetic blood profiles for the pharmaceutical compositions of the present invention were calculated using a simulation program. In this model, PK parameters of the administration of modified release cariprazine formulations at higher doses were predicted based on the 1.5 mg/day single dose administration to healthy volunteers.

Using the formulations and dissolution profiles described in Example 13, as well as the serum concentrations resulting from single administrations of cariprazine, AUC and Cₘₐₓ values were calculated using the pharmacokinetic software, GastroPlus™, in order to predict the impact of physiological and biochemical processes on oral drug bioavailability using modified release formulation in different doses and regimen compared to the corresponding IR doses.

The GastroPlus™ software was used to simulate plasma concentrations of cariprazine at higher doses than applied in the clinical study. GastroPlus™ is an advanced software program that simulates the absorption, pharmacokinetics, and pharmacodynamics for drugs administered via intravenous, oral, ocular, and pulmonary routes in human and preclinical species. The underlying model is the Advanced Compartmental Absorption and Transit (ACAT) model. Since 1997, Simulations Plus has evolved the ACAT model to a high state of refinement, providing the industry's most accurate, flexible, and powerful simulation program.

For model building physicochemical (pKa, solubility-pH data including biorelevant solubilities, logP, permeability across Caco2 cells, particle size of distribution) and biopharmaceutical parameters (time-plasma concentration curve, blood/plasma concentration ratio, fraction unbound in plasma (%)) were measured. To determine pharmacokinetic parameters including clearance, volume of distribution, K12 and K21 rate constants were derived by fitting a two-compartment model on the time-plasma concentration curve (using PKPlus Module in the GastroPlus software), to determine the time-in vivo release (%) profile IVIVC Module in the GastroPlus software was used.

Table 34 shows the results of the GastroPlus™ simulation measured in a 31 days interval.

TABLE 34
GastroPlus ™ simulation (31 days interval)

| | Dosing | PR B | PR B | IR | | | |
|---|---|---|---|---|---|---|---|
| Dose | frequency | Cₘₐₓ | AUC₀₋ₜ | Cₘₐₓ | AUC₀₋ₜ | PRB/IR % | PRB/IR % |
| (mg) | (days) | (ng/ml) | (ng*h/ml) | (ng/ml) | (ng*h/ml) | Cₘₐₓ | AUC |
| 6 | 4 | 2.378 | 1071.3 | 15.19 | 924.79 | 15.66 | 115.84 |
| 6 | 2 | 2.454 | 1328.7 | 23.99 | 923.85 | 10.23 | 143.82 |
| 9 | 2 | 3.580 | 1942.0 | 35.98 | 1384.97 | 9.95 | 140.22 |
| 9 | 3 | 2.587 | 1271.0 | 27.09 | 1385.87 | 9.55 | 91.71 |
| 10.5 | 7 | 3.289 | 1162.6 | 20.24 | 1618.26 | 16.25 | 71.84 |
| 12 | 2 | 4.647 | 2525.8 | 47.96 | 1846.07 | 9.69 | 136.82 |
| 12 | 4 | 4.645 | 2108.0 | 30.37 | 1848.12 | 15.29 | 114.06 |
| 18 | 3 | 4.782 | 2368.3 | 54.14 | 2768.96 | 8.83 | 85.53 |
| 18 | 4 | 6.760 | 3097.1 | 45.54 | 2770.29 | 14.84 | 111.80 |
| 21 | 7 | 6.576 | 2324.8 | 40.47 | 3234.14 | 16.25 | 71.88 |
| 21 | 14 | 5.381 | 4343.4 | 32.22 | 3233.77 | 16.70 | 134.31 |
| 24 | 4 | 8.043 | 3760.1 | 60.71 | 3692.63 | 13.3 | 101.8 |
| 31.5 | 7 | 9.843 | 3482.4 | 60.7 | 4849.28 | 16.22 | 71.81 |
| 42 | 7 | 12.960 | 4608.6 | 80.93 | 6464.15 | 16.01 | 71.29 |

1. An orally deliverable solid pharmaceutical composition for the modified release of cariprazine or pharmaceutically acceptable salts thereof, wherein the composition comprises a therapeutically effective amount of cariprazine or a pharmaceutically acceptable salt thereof and at least one release-modifying agent.

2. The solid pharmaceutical composition of claim 1, wherein the composition comprises a therapeutically effective amount of cariprazine or a pharmaceutically acceptable salt thereof and at least one release-modifying agent suitable for decreasing the Cₘₐₓ and keeping the AUC values within the range of the effective and tolerable therapeutic daily doses aiming an elongated effect in the desired administration frequency independently of the location of the drug release in the gastrointestinal tract.

3. The solid pharmaceutical composition according to claim 1 comprising from about 1.5 mg to about 84 mg cariprazine in the form of a pharmaceutically acceptable salt.

4-8. (canceled)

9. The solid pharmaceutical composition according to claim 1 comprising from about 1.5 mg to about 84 mg cariprazine in the form of a hydrochloride salt.

10-11. (canceled)

12. The solid pharmaceutical composition according to claim 1 comprising a pharmaceutically acceptable salt of cariprazine selected from the group consisting of a salt of hydrochloric acid, sulphuric acid, phosphoric acid, methane sulfonic acid, camphor sulfonic acid, oxalic acid, maleic acid, succinic acid, citric acid, formic acid, hydrobromic acid, benzoic acid, tartaric acid, fumaric acid, salicylic acid, mandelic acid, and carbonic acid.

13. The solid pharmaceutical composition according to claim 12 comprising a pharmaceutically acceptable salt of cariprazine selected from the group consisting of a salt of hydrochloric acid, hydrobromic acid, and methanesulfonic acid.

14. The solid pharmaceutical composition according to claim 1 comprising at least one release-modifying agent selected from the group consisting of hydrophilic and hydrophobic polymers.

15. The solid pharmaceutical composition according to claim 14 comprising at least one hydrophilic polymer as a release-modifying agent.

16. The solid pharmaceutical composition according to claim 14 comprising at least one cellulose-based polymer as a release-modifying agent.

17. The solid pharmaceutical composition according to claim 14 comprising at least one cellulose-based polymer as a release-modifying agent selected from the group consisting of hydroxypropyl cellulose (HPC), hydroxyethyl cellulose (HEC), hydroxymethyl cellulose, hydroxypropyl methylcellulose (HPMC), carboxymethyl cellulose, sodium carboxymethyl cellulose, methylcellulose, and hydroxyethyl methylcellulose.

18. (canceled)

19. The solid pharmaceutical composition according to claim 14 comprising at least one release-modifying agent from about 15 to about 75% by weight.

20. (canceled)

21. The solid pharmaceutical composition according to claim 1 comprising additional excipients alone or in any combination, selected from the group consisting of diluents, lubricants, binders, granulating aids, effervescent components, film formers, and glidants.

22. The solid pharmaceutical composition according to claim 1 in the form of an oral formulation.

23. The solid pharmaceutical composition according to claim 1 exhibiting a dissolution profile, wherein about 25% to about 70% of the total amount of cariprazine is in solution at 4 hours, about 45% to about 100% of the total amount of cariprazine is in solution at 8 hours, and about 65% to about 100% of the total amount of cariprazine is in solution at 12 hours.

24-25. (canceled)

26. The solid pharmaceutical composition according to claim 1 exhibiting a cariprazine AUC value following oral administration that is from about 60% to about 145% of that achieved using an immediate release (IR) dosage form of cariprazine when administered orally at an equivalent dose.

27-30. (canceled)

31. The solid pharmaceutical composition according to claim 1 exhibiting a PK profile after oral administration in a human wherein Cₘₐₓ is from about 8% to about 40% of the Cₘₐₓ obtained by an IR formulation comprising the same amount of cariprazine as said modified release pharmaceutical composition; when said PK profile arises from a PK experiment performed in a human fasted overnight for at least eight hours prior to dosing; wherein said PK profile is based on plasma concentrations of the total cariprazine; and wherein said pharmaceutical composition comprises cariprazine in a therapeutically effective amount.

32-58. (canceled)

59. A method of treating a patient suffering from a pathological condition which requires the modulation of dopamine receptors, wherein the method comprises the administration of the pharmaceutical compositions according to claim 1 less frequent than daily to a patient in need thereof.

60-66. (canceled)

67. The method of treating a patient according to claim 59, wherein the patient is suffering from a pathological condition which require the modulation of dopamine receptors selected from the group consisting of: psychoses drug abuse, cognitive impairment accompanying schizophrenia, mild-to-moderate cognitive deficits, dementia, psychotic states associated with dementia, eating disorders, attention deficit disorders, hyperactivity disorders in children, psychotic depression, mania, paranoid and delusional disorders, dyskinetic disorders, anxiety, sexual dysfunction, sleep disorders, emesis, aggression, and autism.

68. The method of treating a patient according to claim 67, wherein the patient is suffering from schizophrenia and/or mania.
